(19) United States
(12) Patent Application Publication
(10) Pub. No.: US 2024/0158820 A1
(43) Pub. Date: May 16, 2024

(54) FERMENTATIVE PRODUCTION OF N-BUTYLACRYLATE USING ALCOHOL ACYL TRANSFERASE ENZYMES

(71) Applicant: BASF SE, Ludwigshafen am Rhein (DE)

(72) Inventors: Stephan SAUM, Lampertheim (DE); Woncheol KIM, Tarrytown, NY (US); Oskar ZELDER, Ludwigshafen (DE); Jennifer JAITZIG, Ludwigshafen (DE); Zheyuan GUO, San Diego, CA (US)

(21) Appl. No.: 18/419,647

(22) Filed: Jan. 23, 2024

Related U.S. Application Data
(62) Division of application No. 16/089,642, filed on Sep. 28, 2018, now Pat. No. 11,913,054.

(30) Foreign Application Priority Data
Mar. 30, 2016 (EP) 16162887.0

(51) Int. Cl. C12P 7/62; C12N 9/10

(52) U.S. Cl. CPC C12P 7/62; C12N 9/1029; C12Y 203/01084

(57) ABSTRACT

Provided herein is a recombinant nucleic acid molecule, a recombinant microorganism, and a method for fermentative production of n-butylacrylate and other esters from alcohols and acyl-CoA units using alcohol acyl transferase enzymes.

### CROSS-REFERENCE TO RELATED APPLICATIONS

This application is a divisional application of U.S. application Ser. No. 16/089,642, filed Sep. 28, 2018, which is a U.S. National Phase Application of International Patent Application No. PCT/EP17/56872, filed Mar. 22, 2017, which claims the benefit of priority to European Patent Application No. 16162887.0, filed Mar. 30, 2016, the entire contents of which are hereby incorporated by reference herein

### REFERENCE TO AN ELECTRONIC SEQUENCE LISTING

The contents of the electronic sequence listing (DIV sequence listing_27843-2467.xml; Size: 194,086 bytes; and Date of Creation: Oct. 25, 2023) is herein incorporated by reference in its entirety.

### FIELD OF THE INVENTION

The invention is directed to a recombinant nucleic acid molecule, a recombinant microorganism and to a method for fermentative production of n-butylacrylate and other esters from alcohols and acyl-CoA units using alcohol acyl transferase enzymes.

### BACKGROUND

Acrylate esters are among the most versatile monomers for providing performance properties to a wide variety of polymers. Acrylate esters are chemically produced with acrylic acid and alcohols. For instance, n-butylacrylate (n-BA) is produced by esterifying acrylate with butanol.

Production of n-BA in a biological system has not been demonstrated. A pathway for the production of n-BA in a biological system may comprise an acryloyl-CoA biosynthesis pathway and a butanol biosynthesis pathway. In addition, for fermentative production of n-BA, an alcohol acyl transferase (AAT) enzyme, capable of catalysing an esterification reaction between acryloyl-CoA and butanol, forming an ester bond, would need to be present in a production system, such as a microorganism. Currently, multiple pathways have been reported for the production of acryloyl-CoA or butanol in a biological system via natural or engineered pathways (Teufel, Kung et al. 2009) (Schadeweg and Boles 2016).

Also, various publications describe AAT enzyme activities that combine acyl-CoAs with alcohols through ester formation (El-Sharkawy, Manriquez et al. 2005). They show that some AATs have broad substrate specificities resulting in various ester products. However, no AAT enzymes capable of forming n-BA esters in a biological system by esterification of acryloyl-CoA and butanol were described known.

The physiological properties of acryloyl-CoA and butanol and their intermediates limited the functionality and/or the capacity of fermentative production of these products in microorganisms (Zhou, Zhang et al. 2011). Many difficulties, such as reducing power imbalance, toxicity, ATP imbalance, and oxygen sensitivity of enzymes also make the development of engineered strain to produce acryl ester cumbersome.

Especially, acrylate, butanol, and n-BA are toxic to the cells. Although, there are some examples in public for producing acrylate and butanol by fermentation separately in yeast and various other host strains, there is no reported biological method to produce n-butyl acrylate in a biological system due to the lack of adequate AAT enzymes catalysing the esterification reaction.

Today n-BA is mainly produced from chemical methods based on petroleum based feedstock. Availability of a sustainable method for producing acrylate esters is becoming interesting. One way for such a sustainable method could be a biological system for fermentative production of acrylate esters from renewable feed stock, such as glucose or lignocellulose. Fermentative n-BA production is hypothetically possible, if acryloyl-CoA and butanol are produced in a cell and subsequently combined to form n-BA by an AAT enzyme.

Identifying AAT enzymes catalysing this esterification step is a key challenge for providing such fermentative production system.

### SUMMARY

It is therefore one objective of the invention at hand to provide AAT enzymes having an activity of esterifying acryloyl-CoA and butanol to form n-BA. Furthermore, it is an objective of the invention at hand to develop a microorganism capable of fermentative production of n-BA by esterification of acryloyl-CoA and butanol and to provide fermentation systems for the production of n-BA.

It is an additional objective of the invention at hand to provide AAT enzymes having an activity of esterifying propionyl-CoA and butanol to form butylpropionate and/or of esterifying lactoyl-CoA and butanol to form butyl lactate and or of esterifying acetyl-CoA and ethanol to form ethyl acetate. Further it is an objective of the invention at hand to develop microorganisms capable of fermentative production of butyl propionate, butyl lactate and/or ethyl acetate by esterification of propionyl-CoA and butanol, lactoyl-CoA and butanol and/or acetyl-CoA and ethanol, respectively and to provide fermentation systems for the production of butyl propionate, butyl lactate and/or ethyl acetate.

### BRIEF DESCRIPTION OF THE DRAWINGS

FIG. 1 is a diagram of pYP137 construction vector for overexpression of Aco in S. cerevisiae.

FIG. 2 is a diagram of pYP096, pYP106 construction vector for overexpression of pct in S. cerevisiae.

FIG. 3 is a diagram of pYP083 construction vector for overexpression of AAT in S. cerevisiae.

FIGS. 4A and 4B are graphs of nBA and nBP, respectively, production in S. cerevisiae by feeding propionate and butanol. TYC-166 is a test strain with ACO, Pct-Me, and Cm-AAT2 expressed. TYC-181 is a negative control strain with Pct-Me and Cm-AAT2 but without ACO expressed.

### DETAILED DESCRIPTION OF THE INVENTION

One embodiment of the invention is a method for fermentative production of n-butylacrylate (n-BA) comprising the steps of
i) providing a recombinant microorganism comprising a butanol producing pathway, an acryloyl-CoA producing pathway and expressing an AAT gene encoding an AAT enzyme having an n-BA forming activity and
ii) culturing said microorganism under conditions that allow for the production of n-BA and
iii) recovering n-BA from the fermentation broth.

The term AAT enzyme or AAT enzyme activity means an enzyme or an enzyme activity as defined by EC 2.3.1.84, catalysing an esterification step, transferring an acyl-CoA to an alcohol. An AAT enzyme having an n-BA forming activity means an AAT enzyme catalysing the reaction of acryloyl-CoA and butanol to n-BA and CoA. The AAT enzyme may be endogenous or heterologous to the microorganism.

A “butanol producing pathway” means a metabolic pathway comprising all enzymes catalysing the biochemical reactions to form butanol from other metabolite(s). Such pathways have previously been established in microorganisms as for example described in (Schadeweg and Boles 2016).

A “acryloyl-CoA producing pathway” means a metabolic pathway comprising all enzymes catalyzing the biochemical reactions to form acryloyl-CoA from other metabolite(s). Such pathways have previously been established in microorganisms as for example described in (Zhou, Zhang et al. 2011) (Chu, Ahn et al. 2015).

Another embodiment of the invention is a method for fermentative production of n-BA as described above wherein the AAT gene encoding an AAT enzyme having an n-BA forming activity is selected from the group consisting of
(I) a nucleic acid molecule comprising a sequence of SEQ ID NO: 1, 3, 5, 7, 9, 11 or 13, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11 or 13, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11 or 13, and
(IV) a nucleic acid molecule encoding a polypeptide of SEQ ID NO: 2, 4, 6, 8, 10, 12, or 14 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, or 14 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the n-BA forming activity as the polypeptide having SEQ ID NO: 2, 4, 6, 8, 10, 12, or 14.

n-BA forming activity may be quantified by an assay as described in (Fikri E. L. Yahyaoui 2002).

A further embodiment of the invention is a recombinant microorganism comprising an introduced, increased or enhanced activity and/or expression of a nucleic acid molecule encoding an AAT gene encoding an AAT enzyme having an n-BA forming activity.

Preferably, said recombinant microorganism is further comprising a butanol producing pathway and an acryloyl-CoA producing pathway. More preferably, the nucleic acid molecule encoding an AAT gene encoding an AAT enzyme having an n-BA forming activity that is having an introduced, increased or enhanced activity and/or expression in the recombinant microorganism of the invention is selected from the group consisting of
(I) a nucleic acid molecule comprising a sequence of SEQ ID NO: 1, 3, 5, 7, 9, 11 or 13, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11 or 13, and
(III) a nucleic acid molecule hybridizing under medium stringent conditions, more preferably under high stringent conditions, most preferably under very high stringent conditions to the complement of a nucleic acid molecule having SEQ ID NO: 1, 3, 5, 7, 9, 11 or 13, and
(IV) a nucleic acid molecule encoding a polypeptide of SEQ ID NO: 2, 4, 6, 8, 10, 12, or 14 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, or 14 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the n-BA forming activity as the polypeptide having SEQ ID NO: 2, 4, 6, 8, 10, 12, or 14.

In one embodiment the n-BA forming recombinant microorganism of the invention is selected from the group of prokaryotic microorganisms comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Clostridium propionicum, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Lactobacillus spp., Micrococcus sp. CCM825, Morganella morganii, Nocardia opaca, Nocardia rugosa, Pantoea agglomerans, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp, Zymomonas mobilis and so forth.

In another embodiment of the invention, the microorganism is a eukaryotic cell. Suitable eukaryotic cells include yeast cells, as for example Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia or Trichosporon spp., Yamadazyma spp. or Pseudozyma spp.

Preferably, the microorganism is a yeast of the genus Saccharomyces spec, most preferably Saccharomyces cerevisiae TYC-072 [MATa; ura3-52; trp1-289; leu2-3_112; his3 Δ1; MAL2-8C; SUC2 adh1::IoxP adh3::IoxP; adh4Δ::IoxP, adh5Δ::IoxP Δadh1,3,4,5 (all with IoxP)].

A further embodiment of the invention is a composition comprising one or more recombinant n-BA forming microorganisms of the invention as defined above. The composition may further comprise a medium that allows grow of the recombinant microorganism of the invention. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. (Steen, Chan et al. 2008, Krivoruchko, Serrano-Amatriain et al. 2013, Tang, Feng et al. 2013). Preferably the carbon source is glucose or sucrose, more preferably the carbon source is glucose.

A further embodiment of the invention is a method for producing a recombinant microorganism producing n-BA comprising the steps of:
(I) introducing, increasing or enhancing the activity and/or expression of an AAT gene encoding an AAT enzyme having an n-BA forming activity in a microorganism; and
(II) further introducing in the microorganism a butanol producing pathway and an acryloyl-CoA producing pathway.

Introducing a butanol or an acryloyl-CoA producing pathway means introducing into the recombinant microorganism all enzymes necessary for catalysing the biochemical reactions to form butanol or acryloyl-CoA respectively, from other metabolite(s).

The AAT gene that is introduced, increased or enhanced in the recombinant microorganism used in the method for producing a recombinant microorganism producing n-BA is selected from the group consisting of
(I) a nucleic acid molecule comprising a sequence of SEQ ID NO: 1, 3, 5, 7, 9, 11 or 13, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11 or 13, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11 or 13, and
(IV) a nucleic acid molecule encoding a polypeptide of SEQ ID NO: 2, 4, 6, 8, 10, 12, or 14 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, or 14 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the n-BA forming activity as the polypeptide having SEQ ID NO: 2, 4, 6, 8, 10, 12, or 14.

The microorganism may be selected from the group of microorganisms as defined above.

A further embodiment of the invention is a recombinant expression construct or a recombinant vector comprising said recombinant expression construct wherein the recombinant expression construct is comprising a promoter functional in a microorganism functionally linked to a nucleic acid molecule having a sequence selected from the group consisting of
(I) a nucleic acid molecule comprising a sequence of SEQ ID NO: 1, 3, 5, 7, 9, 11 or 13, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11 or 13, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11 or 13, and
(IV) a nucleic acid molecule encoding a polypeptide of SEQ ID NO: 2, 4, 6, 8, 10, 12, or 14 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, or 14 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the n-BA forming activity as the polypeptide having SEQ ID NO: 2, 4, 6, 8, 10, 12, or 14 and wherein the promoter is heterologous to said nucleic acid molecule.

A further embodiment of the invention is a method of culturing or growing the n-BA forming recombinant microorganism of the invention comprising the steps of inoculating a culture medium with one or more recombinant microorganism of the invention and culturing or growing said recombinant microorganism in culture medium. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. Preferably the carbon source is glucose or sucrose, more preferably the carbon source is glucose.

In some embodiments, the n-BA forming recombinant microorganisms encompassed by the invention are grown under batch or continuous fermentations conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation which also finds use in the present invention is a system where a defined fermentation medium is added continuously to a bioreactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out in a temperature within the range of from about 10° C. to about 60° C., from about 15° C. to about 50° C., from about 20° C. to about 45° C., from about 25° C. to about 45° C., from about 30° C. to about 45° C. and from about 25° C. to about 40° C. In a preferred embodiment the temperature is about 28° C., 30° C. or 32° C. In a most preferred embodiment the temperature is about 28° C. or 30° C.

In some other embodiments, the fermentation is carried out for a period of time within the range of from about 8 hours to 240 hours, from about 8 hours to about 168 hours, from about 10 hours to about 144 hours, from about 15 hours to about 120 hours, or from about 20 hours to about 72 hours. Preferably the fermentation is carried out from about 20 hours to about 40 hours.

In some other embodiments, the fermentation is carried out at a pH in the range of about 4 to about 9, in the range of about 4.5 to about 8.5, in the range of about 5 to about 8, or in the range of about 5.5 to about 7.5. Preferably the fermentation will be carried out at a pH of 5-6.

In one embodiment of the method of producing n-BA, the microorganism is cultured in a medium comprising between 1% and 30% (w/v) of a sugar, between 5% and 25% (w/v) of a sugar, between 10% and 20% (w/v) of a sugar, between 14% and 18% (w/v) of a sugar. Preferably the microorganism is cultured in a medium comprising between 15% and 20% (w/v) of a sugar.

A use of a n-BA forming recombinant microorganism of the invention or a composition of the invention for the fermentative production of n-BA is an additional embodiment of the invention. A further embodiment of the invention is a process for fermentative production of n-BA comprising the steps of
I) growing the n-BA forming microorganism as defined above in a fermenter and
II) recovering n-BA from the fermentation broth obtained in I).

A further embodiment of the invention is a method for fermentative production of butyl propionate comprising the steps of
i) providing a recombinant microorganism comprising a butanol producing pathway, an propionyl-CoA producing pathway and expressing an AAT gene encoding an AAT enzyme having an butyl propionate forming activity and
ii) culturing said microorganism under conditions that allow for the production of butyl propionate and
iii) recovering butyl propionate from the fermentation broth.

The term AAT enzyme or AAT enzyme activity means an enzyme or an enzyme activity as defined by EC 2.3.1.84, catalysing an esterification step, transferring an acyl-CoA to an alcohol. An AAT enzyme having a butyl propionate forming activity means an AAT enzyme catalysing the reaction of propionyl-CoA and butanol to butyl propionate and CoA. The AAT enzyme may be endogenous or heterologous to the microorganism.

A “butanol producing pathway” means a metabolic pathway comprising all enzymes catalysing the biochemical reactions to form butanol from other metabolite(s). Such pathways have previously been established in microorganisms as for example described in (Schadeweg and Boles 2016).

A “propionyl-CoA producing pathway” means a metabolic pathway comprising all enzymes catalyzing the biochemical reactions to form propionyl-CoA from other metabolite(s). Such pathways have previously been established in microorganisms as for example described in (Yuzawa, Chiba et al. 2012).

Another embodiment of the invention is a method for fermentative production of butyl propionate wherein the AAT gene encoding an AAT enzyme having a butyl propionate forming activity is selected from the group consisting of
(I) a nucleic acid molecule comprising a sequence of SEQ ID NO: 1, 3, 5, 7, 9, 11, 13, 15, 17, 19, 21, 23 or 25, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11, 13, 15, 17, 19, 21, 23 or 25, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11, 13, 15, 17, 19, 21, 23 or 25, and
(IV) a nucleic acid molecule encoding a polypeptide of SEQ ID NO: 2, 4, 6, 8, 10, 12, 14, 16, 18, 20, 22, 24 or 26 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, 14, 16, 18, 20, 22, 24 or 26 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 16, 18, 20, 22, 24 or 26.

Butyl propionate forming activity may be quantified by an assay as described in (Fikri E. L. Yahyaoui 2002).

A further embodiment of the invention is a butyl propionate forming recombinant microorganism comprising an introduced, increased or enhanced activity and/or expression of a nucleic acid molecule encoding an AAT gene encoding an AAT enzyme having a butyl propionate forming activity. Preferably, said recombinant microorganism is further comprising a butanol producing pathway and an propionyl-CoA producing pathway. More preferably, the nucleic acid molecule encoding an AAT gene encoding an AAT enzyme having a butyl propionate forming activity that is having an introduced, increased or enhanced activity and/or expression in the recombinant microorganism of the invention is selected from the group consisting of
(I) a nucleic acid molecule comprising a sequence of SEQ ID NO: 1, 3, 5, 7, 9, 11, 13, 15, 17, 19, 21, 23 or 25, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11, 13, 15, 17, 19, 21, 23 or 25, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11, 13, 15, 17, 19, 21, 23 or 25, and
(IV) a nucleic acid molecule encoding a polypeptide of SEQ ID NO: 2, 4, 6, 8, 10, 12, 14, 16, 18, 20, 22, 24 or 26 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, 14, 16, 18, 20, 22, 24 or 26 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 16, 18, 20, 22, 24 or 26,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 16, 18, 20, 22, 24 or 26.

In one embodiment the butyl propionate forming recombinant microorganism of the invention is selected from the group of prokaryotic microorganisms comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Clostridium propionicum, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Lactobacillus spp., Micrococcus sp. CCM825, Morganella morganii, Nocardia opaca, Nocardia rugosa, Pantoea agglomerans, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. spaericum, Nostoc punctiforme, Spirulina platensis, Lyngbya majuscula, L. lagerheimii, Phormidium tenue, Anabaena sp., Leptolyngbya sp, Zymomonas mobilis and so forth.

In another embodiment of the invention, the microorganism is a eukaryotic cell. Suitable eukaryotic cells include yeast cells, as for example Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia or Trichosporon spp., Yamadazyma spp. or Pseudozyma spp.

Preferably, the microorganism is a yeast of the genus Saccharomyces spec, most preferably Saccharomyces cerevisiae TYC-072 [MATa; ura3-52; trp1-289; leu2-3_112; his3 Δ1; MAL2-8C; SUC2 adh1::IoxP adh3::IoxP; adh4Δ::IoxP, adh5Δ:IoxP Δadh1,3,4,5 (all with IoxP)].

A further embodiment of the invention is a composition comprising one or more recombinant butyl propionate forming microorganisms of the invention. The composition may further comprise a medium that allows grow of the recombinant microorganism of the invention. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. (Steen, Chan et al. 2008, Krivoruchko, Serrano-Amatriain et al. 2013, Tang, Feng et al. 2013). Preferably the carbon source is glucose or sucrose, more preferably the carbon source is glucose.

A further embodiment of the invention is a method for producing a recombinant microorganism producing butyl propionate comprising the steps of:
(I) introducing, increasing or enhancing the activity and/or expression of an AAT gene encoding an AAT enzyme having an butyl propionate forming activity in a microorganism; and
(II) further introducing in the microorganism a butanol producing pathway and an propionyl-CoA producing pathway.

Introducing a butanol or a propionyl-CoA producing pathway means introducing into the recombinant microorganism all enzymes necessary for catalysing the biochemical reactions to form butanol or propionyl-CoA respectively, from other metabolite(s).

The AAT gene that is introduced, increased or enhanced in the recombinant microorganism used in the method for producing a recombinant microorganism producing butyl propionate is selected from the group consisting of
(I) a nucleic acid molecule comprising a sequence of SEQ ID NO: 1, 3, 5, 7, 9, 11, 13, 15, 17, 19, 21, 23 or 25, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11, 13, 15, 17, 19, 21, 23 or 25, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11, 13, 15, 17, 19, 21, 23 or 25, and
(IV) a nucleic acid molecule encoding a polypeptide of SEQ ID NO: 2, 4, 6, 8, 10, 12, 14, 16, 18, 20, 22, 24 or 26 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, 14, 16, 18, 20, 22, 24 or 26 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 40 16, 18, 20, 22, 24 or 26,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 16, 18, 20, 22, 24 or 26.

The microorganism may be selected from the group of microorganisms as defined above.

A further embodiment of the invention is a recombinant expression construct or a recombinant vector comprising said recombinant expression construct wherein the recombinant expression construct is comprising a promoter functional in a microorganism functionally linked to a nucleic acid molecule having a sequence selected from the group consisting of
(I) a nucleic acid molecule comprising a sequence of SEQ ID NO: 1, 3, 5, 7, 9, 11, 13, 15, 17, 19, 21, 23 or 25, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 11, 13, 15, 17, 19, 21, 23 or 25, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 11, 13, 15, 17, 19, 21, 23 or 25, and
(IV) a nucleic acid molecule encoding a polypeptide of SEQ ID NO: 2, 4, 6, 8, 10, 12, 14, 16, 18, 20, 22, 24 or 26 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 12, 14, 16, 18, 20, 22, 24 or 26 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl propionate forming activity as the polypeptide having SEQ ID NO: 2, 4, 6, 8, 10, 12, 14, 16, 18, 20, 22, 24 or 26 and wherein the promoter is heterologous to said nucleic acid molecule.

A further embodiment of the invention is a method of culturing or growing the butyl propionate forming recombinant microorganism of the invention comprising the steps of inoculating a culture medium with one or more recombinant microorganism of the invention and culturing or growing said recombinant microorganism in culture medium. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. Preferably the carbon source is glucose or sucrose, more preferably the carbon source is glucose.

In some embodiments, the butyl propionate forming recombinant microorganisms encompassed by the invention are grown under batch or continuous fermentations conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation which also finds use in the present invention is a system where a defined fermentation medium is added continuously to a bioreactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out in a temperature within the range of from about 10° C. to about 60° C., from about 15° C. to about 50° C., from about 20° C. to about 45° C., from about 25° C. to about 45° C., from about 30° C. to about 45° C. and from about 25° C. to about 40° C. In a preferred embodiment the temperature is about 28° C., 30° C. or 32° C. In a most preferred embodiment the temperature is about 28° C. or 30° C.

In some other embodiments, the fermentation is carried out for a period of time within the range of from about 8 hours to 240 hours, from about 8 hours to about 168 hours, from about 10 hours to about 144 hours, from about 15 hours to about 120 hours, or from about 20 hours to about 72 hours. Preferably the fermentation is carried out from about 20 hours to about 40 hours.

In some other embodiments, the fermentation is carried out at a pH in the range of about 4 to about 9, in the range of about 4.5 to about 8.5, in the range of about 5 to about 8, or in the range of about 5.5 to about 7.5. Preferably the fermentation will be carried out at a pH of 5-6.

In one embodiment of the method of producing butyl propionate, the microorganism is cultured in a medium comprising between 1% and 30% (w/v) of a sugar, between 5% and 25% (w/v) of a sugar, between 10% and 20% (w/v) of a sugar, between 14% and 18% (w/v) of a sugar. Preferably the microorganism is cultured in a medium comprising between 15% and 20% (w/v) of a sugar.

A use of a butyl propionate forming recombinant microorganism of the invention or a composition of the invention for the fermentative production of butyl propionate is an additional embodiment of the invention. A further embodiment of the invention is a process for fermentative production of butyl propionate comprising the steps of
I) growing the butyl propionate forming microorganism of the invention in a fermenter and
II) recovering butyl propionate from the fermentation broth obtained in I).

A further embodiment of the invention is a method for fermentative production of butyl lactate comprising the steps of
i) providing a recombinant microorganism comprising a butanol producing pathway, a lactoyl-CoA producing pathway and expressing an AAT gene encoding an AAT enzyme having a butyl lactate forming activity and
ii) culturing said microorganism under conditions that allow for the production of butyl lactate and
iii) recovering butyl lactate from the fermentation broth.

The term AAT enzyme or AAT enzyme activity means an enzyme or an enzyme activity as defined by EC 2.3.1.84, catalysing an esterification step, transferring an acyl-CoA to an alcohol. An AAT enzyme having a butyl lactate forming activity means an AAT enzyme catalysing the reaction of lactoyl-CoA and butanol to butyl lactate and CoA. The AAT enzyme may be endogenous or heterologous to the microorganism.

A “butanol producing pathway” means a metabolic pathway comprising all enzymes catalysing the biochemical reactions to form butanol from other metabolite(s). Such pathways have previously been established in microorganisms as for example described in (Schadeweg and Boles 2016).

A “lactoyl-CoA producing pathway” means a metabolic pathway comprising all enzymes catalyzing the biochemical reactions to form lactoyl-CoA from other metabolite(s). Such pathways have previously been established in microorganisms as for example described in (Nevoigt 2008).

Another embodiment of the invention is a method for fermentative production of butyl lactate wherein the AAT gene encoding an AAT enzyme having a butyl lactate forming activity is selected from the group consisting of
(I) a nucleic acid molecule comprising a sequence of SEQ ID NO: 1, 3, 5, 7, 9, 15, 17 or 19 and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 15, 17 or 19, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 15, 17 or 19, and
(IV) a nucleic acid molecule encoding a polypeptide of SEQ ID NO: 2, 4, 6, 8, 10, 16, 18 or 20 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO:: 2, 4, 6, 8, 10, 16, 18 or 20 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl lactate forming activity as the polypeptide having SEQ ID NO:: 2, 4, 6, 8, 10, 16, 18 or 20.

Butyl lactate forming activity may be quantified by an assay as described in (Fikri E. L. Yahyaoui 2002).

A further embodiment of the invention is a butyl lactate forming recombinant microorganism comprising an introduced, increased or enhanced activity and/or expression of a nucleic acid molecule encoding an AAT gene encoding an AAT enzyme having a butyl lactate forming activity. Preferably, said recombinant microorganism is further comprising a butanol producing pathway and a lactoyl-CoA producing pathway. More preferably, the nucleic acid molecule encoding an AAT gene encoding an AAT enzyme having a butyl lactate forming activity that is having an introduced, increased or enhanced activity and/or expression in the recombinant microorganism of the invention is selected from the group consisting of
(I) a nucleic acid molecule comprising a sequence of SEQ ID NO: 1, 3, 5, 7, 9, 15, 17 or 19 and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 15, 17 or 19, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 15, 17 or 19, and
(IV) a nucleic acid molecule encoding a polypeptide of SEQ ID NO: 2, 4, 6, 8, 10, 16, 18 or 20 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 16, 18 or 20 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl lactate forming activity as the polypeptide having SEQ ID NO: 2, 4, 6, 8, 10, 16, 18 or 20.

In one embodiment the butyl lactate forming recombinant microorganism of the invention is selected from the group of prokaryotic microorganisms comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Clostridium propionicum, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Lactobacillus spp., Micrococcus sp. CCM825, Morganella morganii, Nocardia opaca, Nocardia rugosa, Pantoea agglomerans, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. spaericum, Nostoc punctiforme, Spirulina platensis, Lyngbya majuscula, L. lagerheimii, Phormidium tenue, Anabaena sp., Leptolyngbya sp, Zymomonas mobilis and so forth.

In another embodiment of the invention, the microorganism is a eukaryotic cell. Suitable eukaryotic cells include yeast cells, as for example Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia or Trichosporon spp., Yamadazyma spp. or Pseudozyma spp.

Preferably, the microorganism is a yeast of the genus Saccharomyces spec, most preferably Saccharomyces cerevisiae TYC-072 [MATa; ura3-52; trp1-289; leu2-3_112; his3 Δ1; MAL2-8C; SUC2 adh1::IoxP adh3::IoxP; adh4Δ::IoxP, adh5Δ::IoxP Δadh 1,3,4,5 (all with IoxP)].

A further embodiment of the invention is a composition comprising one or more recombinant butyl lactate forming microorganisms of the invention. The composition may further comprise a medium that allows grow of the recombinant microorganism of the invention. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. (Steen, Chan et al. 2008, Krivoruchko, Serrano-Amatriain et al. 2013, Tang, Feng et al. 2013). Preferably the carbon source is glucose or sucrose, more preferably the carbon source is glucose.

A further embodiment of the invention is a method for producing a recombinant microorganism producing butyl lactate comprising the steps of:
(I) introducing, increasing or enhancing the activity and/or expression of an AAT gene encoding an AAT enzyme having a butyl lactate forming activity in a microorganism; and
(II) further introducing in the microorganism a butanol producing pathway and a lactoyl-CoA producing pathway.

Introducing a butanol or a lactoyl-CoA producing pathway means introducing into the recombinant microorganism all enzymes necessary for catalysing the biochemical reactions to form butanol or lactoyl-CoA respectively, from other metabolite(s).

The AAT gene that is introduced, increased or enhanced in the recombinant microorganism used in the method for producing a recombinant microorganism producing butyl lactate is selected from the group consisting of
(I) a nucleic acid molecule comprising a sequence of SEQ ID NO: 1, 3, 5, 7, 9, 15, 17 or 19 and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 15, 17 or 19, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 15, 17 or 19, and
(IV) a nucleic acid molecule encoding a polypeptide of SEQ ID NO: 2, 4, 6, 8, 10, 16, 18 or 20 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 16, 18 or 20 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl lactate forming activity as the polypeptide having SEQ ID NO: 2, 4, 6, 8, 10, 16, 18 or 20.

The microorganism may be selected from the group of microorganisms as defined above.

A further embodiment of the invention is a recombinant expression construct or a recombinant vector comprising said recombinant expression construct wherein the recombinant expression construct is comprising a promoter functional in a microorganism functionally linked to a nucleic acid molecule having a sequence selected from the group consisting of
(I) a nucleic acid molecule comprising a sequence of SEQ ID NO: 1, 3, 5, 7, 9, 15, 17 or 19 and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 1, 3, 5, 7, 9, 15, 17 or 19, and
(III) a nucleic acid molecule hybridizing under medium stringent conditions, preferably under high stringent conditions, more preferably under very high stringent conditions to the complement of a nucleic acid molecule having SEQ ID NO: 1, 3, 5, 7, 9, 15, 17 or 19, and
(IV) a nucleic acid molecule encoding a polypeptide of SEQ ID NO: 2, 4, 6, 8, 10, 16, 18 or 20 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 2, 4, 6, 8, 10, 16, 18 or 20 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the butyl lactate forming activity as the polypeptide having SEQ ID NO: 2, 4, 6, 8, 10, 16, 18 or 20 and wherein the promoter is heterologous to said nucleic acid molecule.

A further embodiment of the invention is a method of culturing or growing the butyl lactate forming recombinant microorganism of the invention comprising the steps of inoculating a culture medium with one or more recombinant microorganism of the invention and culturing or growing said recombinant microorganism in culture medium. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. Preferably the carbon source is glucose or sucrose, more preferably the carbon source is glucose.

In some embodiments, the butyl lactate forming recombinant microorganisms encompassed by the invention are grown under batch or continuous fermentations conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation which also finds use in the present invention is a system where a defined fermentation medium is added continuously to a bioreactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out in a temperature within the range of from about 10° C. to about 60° C., from about 15° C. to about 50° C., from about 20° C. to about 45° C., from about 25° C. to about 45° C., from about 30° C. to about 45° C. and from about 25° C. to about 40° C. In a preferred embodiment the temperature is about 28° C., 30° C. or 32° C. In a most preferred embodiment the temperature is about 28° C. or 30° C.

In some other embodiments, the fermentation is carried out for a period of time within the range of from about 8 hours to 240 hours, from about 8 hours to about 168 hours, from about 10 hours to about 144 hours, from about 15 hours to about 120 hours, or from about 20 hours to about 72 hours. Preferably the fermentation is carried out from about 20 hours to about 40 hours.

In some other embodiments, the fermentation is carried out at a pH in the range of about 4 to about 9, in the range of about 4.5 to about 8.5, in the range of about 5 to about 8, or in the range of about 5.5 to about 7.5. Preferably the fermentation will be carried out at a pH of 5-6.

In one embodiment of the method of producing butyl lactate, the microorganism is cultured in a medium comprising between 1% and 30% (w/v) of a sugar, between 5% and 25% (w/v) of a sugar, between 10% and 20% (w/v) of a sugar, between 14% and 18% (w/v) of a sugar. Preferably the microorganism is cultured in a medium comprising between 15% and 20% (w/v) of a sugar.

A use of a butyl lactate forming recombinant microorganism of the invention or a composition of the invention for the fermentative production of butyl lactate is an additional embodiment of the invention. A further embodiment of the invention is a process for fermentative production of butyl lactate comprising the steps of
I) growing the butyl lactate forming microorganism of the invention in a fermenter and
II) recovering butyl lactate from the fermentation broth obtained in I).

A further embodiment of the invention is a method for fermentative production of ethyl acetate comprising the steps of
i) providing a recombinant microorganism comprising an ethanol producing pathway, an acetyl-CoA producing pathway and expressing an AAT gene encoding an AAT enzyme having an ethyl acetate forming activity and
ii) culturing said microorganism under conditions that allow for the production of ethyl acetate and
iii) recovering ethyl acetate from the fermentation broth.

The term AAT enzyme or AAT enzyme activity means an enzyme or an enzyme activity as defined by EC 2.3.1.84, catalysing an esterification step, transferring an acyl-CoA to an alcohol. An AAT enzyme having an ethyl acetate forming activity means an AAT enzyme catalysing the reaction of acetyl-CoA and ethanol to ethyl acetate and CoA. The AAT enzyme may be endogenous or heterologous to the microorganism.

An “ethanol producing pathway” means a metabolic pathway comprising all enzymes catalysing the biochemical reactions to form ethanol from other metabolite(s). Such pathways have previously been established in microorganisms (Oeser, 2015, Yeast, 32(1)).

An “acetyl-CoA producing pathway” means a metabolic pathway comprising all enzymes catalyzing the biochemical reactions to form acetyl-CoA from other metabolite(s). Such pathways have previously been established in microorganisms (Schadeweg, V. and E. Boles, 2016, BIOTECHNOLOGY FOR BIOFUELS (9)).

Another embodiment of the invention is a method for fermentative production of ethyl acetate wherein the AAT gene encoding an AAT enzyme having an ethyl acetate forming activity is selected from the group consisting of
(I) a nucleic acid molecule comprising a sequence of SEQ ID NO: 3, 5, 7, 9, 11, 13, 15, 17, 19, 21, 23, 25 or 27, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 3, 5, 7, 9, 11, 13, 15, 17, 19, 21, 23, 25 or 27, and
(III) a nucleic acid molecule hybridizing under medium stringent conditions, preferably under high stringent conditions, more preferably under very high stringent conditions to the complement of a nucleic acid molecule having SEQ ID NO: 3, 5, 7, 9, 11, 13, 15, 17, 19, 21, 23, 25 or 27, and
(IV) a nucleic acid molecule encoding a polypeptide of SEQ ID NO: 4, 6, 8, 10, 12, 14, 16, 18, 20, 22, 24, 26 or 28 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 4, 6, 8, 10, 12, 14, 16, 18, 20, 22, 24, 26 or 28 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the ethyl acetate forming activity as the polypeptide having SEQ ID NO: 4, 6, 8, 10, 12, 14, 16, 18, 20, 22, 24, 26 or 28.

Ethyl acetate forming activity may be quantified by an assay as described in (Fikri E. L. Yahyaoui 2002).

A further embodiment of the invention is an ethyl acetate forming recombinant microorganism comprising an introduced, increased or enhanced activity and/or expression of a nucleic acid molecule encoding an AAT gene encoding an AAT enzyme having an ethyl acetate forming activity. Preferably, said recombinant microorganism is further comprising an ethanol producing pathway and a ethyl-CoA producing pathway. More preferably, the nucleic acid molecule encoding an AAT gene encoding an AAT enzyme having an ethyl acetate forming activity that is having an introduced, increased or enhanced activity and/or expression in the recombinant microorganism of the invention is selected from the group consisting of
(I) a nucleic acid molecule comprising a sequence of SEQ ID NO: 3, 5, 7, 9, 11, 13, 15, 17, 19, 21, 23, 25 or 27, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 3, 5, 7, 9, 11, 13, 15, 17, 19, 21, 23, 25 or 27, and
(III) a nucleic acid molecule hybridizing under medium stringent conditions, preferably under high stringent conditions, more preferably under very high stringent conditions to the complement of a nucleic acid molecule having SEQ ID NO: 3, 5, 7, 9, 11, 13, 15, 17, 19, 21, 23, 25 or 27, and
(IV) a nucleic acid molecule encoding a polypeptide of SEQ ID NO: 4, 6, 8, 10, 12, 14, 16, 18, 20, 22, 24, 26 or 28 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 4, 6, 8, 10, 12, 14, 16, 18, 20, 22, 24, 26 or 28 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the ethyl acetate forming activity as the polypeptide having SEQ ID NO: 4, 6, 8, 10, 12, 14, 16, 18, 20, 22, 24, 26 or 28.

In one embodiment the ethyl acetate forming recombinant microorganism of the invention is selected from the group of prokaryotic microorganisms comprising, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Clostridium propionicum, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Lactobacillus spp., Micrococcus sp. CCM825, Morganella morganii, Nocardia opaca, Nocardia rugosa, Pantoea agglomerans, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., Leptolyngbya sp, Zymomonas mobilis and so forth.

In another embodiment of the invention, the microorganism is a eukaryotic cell. Suitable eukaryotic cells include yeast cells, as for example Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Klebsiella spec, such as Klebsiella pneumonia or Trichosporon spp., Yamadazyma spp. or Pseudozyma spp.

Preferably, the microorganism is a yeast of the genus Saccharomyces spec, most preferably Saccharomyces cerevisiae TYC-072 [MATa; ura3-52; trp1-289; leu2-3_112; his3 Δ1; MAL2-8C; SUC2 adh1::IoxP adh3::IoxP; adh4Δ::IoxP, adh5Δ::IoxP Δadh1,3,4,5 (all with IoxP)].

A further embodiment of the invention is a composition comprising one or more recombinant ethyl acetate forming microorganisms of the invention. The composition may further comprise a medium that allows grow of the recombinant microorganism of the invention. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. (Steen, Chan et al. 2008, Krivoruchko, Serrano-Amatriain et al. 2013, Tang, Feng et al. 2013). Preferably the carbon source is glucose or sucrose, more preferably the carbon source is glucose.

A further embodiment of the invention is a method for producing a recombinant microorganism producing ethyl acetate comprising the steps of:
(I) introducing, increasing or enhancing the activity and/or expression of an AAT gene encoding an AAT enzyme having an ethyl acetate forming activity in a microorganism; and
(II) further introducing in the microorganism an ethanol producing pathway and an acetyl-CoA producing pathway.

Introducing an ethanol or an acetyl-CoA producing pathway means introducing into the recombinant microorganism all enzymes necessary for catalysing the biochemical reactions to form ethanol or acetyl-CoA respectively, from other metabolite(s).

The AAT gene that is introduced, increased or enhanced in the recombinant microorganism used in the method for producing a recombinant microorganism producing ethyl acetate is selected from the group consisting of
(I) a nucleic acid molecule comprising a sequence of SEQ ID NO: 3, 5, 7, 9, 11, 13, 15, 17, 19, 21, 23, 25 or 27, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 3, 5, 7, 9, 11, 13, 15, 17, 19, 21, 23, 25 or 27, and
(III) a nucleic acid molecule hybridizing under medium stringent conditions, preferably under high stringent conditions, more preferably under very high stringent conditions to the complement of a nucleic acid molecule having SEQ ID NO: 3, 5, 7, 9, 11, 13, 15, 17, 19, 21, 23, 25 or 27, and
(IV) a nucleic acid molecule encoding a polypeptide of SEQ ID NO: 4, 6, 8, 10, 12, 14, 16, 18, 20, 22, 24, 26 or 28 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 4, 6, 8, 10, 12, 14, 16, 18, 20, 22, 24, 26 or 28 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the ethyl acetate forming activity as the polypeptide having SEQ ID NO: 4, 6, 8, 10, 12, 14, 16, 18, 20, 22, 24, 26 or 28.

The microorganism may be selected from the group of microorganisms as defined above.

A further embodiment of the invention is a recombinant expression construct or a recombinant vector comprising said recombinant expression construct wherein the recombinant expression construct is comprising a promoter functional in a microorganism functionally linked to a nucleic acid molecule having a sequence selected from the group consisting of
(I) a nucleic acid molecule comprising a sequence of SEQ ID NO: 3, 5, 7, 9, 11, 13, 15, 17, 19, 21, 23, 25 or 27, and
(II) a nucleic acid molecule having at least 80%, preferably at least 85% for example at least 90%, more preferably at least 95% for example at least 96%, even more preferably at least 97% for example at least 98%, most preferably at least 99% identity to a nucleic acid molecule of SEQ ID NO: 3, 5, 7, 9, 11, 13, 15, 17, 19, 21, 23, 25 or 27, and
(III) a nucleic acid molecule hybridizing under medium stringent conditions, preferably under high stringent conditions, more preferably under very high stringent conditions to the complement of a nucleic acid molecule having SEQ ID NO: 3, 5, 7, 9, 11, 13, 15, 17, 19, 21, 23, 25 or 27, and
(IV) a nucleic acid molecule encoding a polypeptide of SEQ ID NO: 4, 6, 8, 10, 12, 14, 16, 18, 20, 22, 24, 26 or 28 or a functional fragment thereof, and
(V) a nucleic acid molecule encoding a polypeptide having at least 60% preferably at least 70% for example at least 75%, more preferably at least 80% for example at least 85%, even more preferably at least 90% for example at least 95%, most preferably at least 96%, at least 97%, at least 98% or at least 99% identity to a polypeptide of SEQ ID NO: 4, 6, 8, 10, 12, 14, 16, 18, 20, 22, 24, 26 or 28 or a functional fragment thereof,
wherein the polypeptide encoded by (II), (III) or (V) is having at least 10% or 20% preferably at least 30% or 50%, more preferably at least 60% or 70%, even more preferably at least 75%, 80%, 85% or 90%, most preferred at least 95% of the ethyl acetate forming activity as the polypeptide having SEQ ID NO: 4, 6, 8, 10, 12, 14, 16, 18, 20, 22, 24, 26 or 28 and wherein the promoter is heterologous to said nucleic acid molecule.

A further embodiment of the invention is a method of culturing or growing the ethyl acetate forming recombinant microorganism of the invention comprising the steps of inoculating a culture medium with one or more recombinant microorganism of the invention and culturing or growing said recombinant microorganism in culture medium. The medium may comprise a carbon source such as hexoses, pentoses or polyols for example sucrose, glucose, fructose, galactose, mannose, raffinose, xylose, arabinose, xylulose, glycerol, mannitol, arabitol, xylitol, starch, cellulose, lignocellulose or combinations thereof. Preferably the carbon source is glucose or sucrose, more preferably the carbon source is glucose.

In some embodiments, the ethyl acetate forming recombinant microorganisms encompassed by the invention are grown under batch or continuous fermentations conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation which also finds use in the present invention is a system where a defined fermentation medium is added continuously to a bioreactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out in a temperature within the range of from about 10° C. to about 60° C., from about 15° C. to about 50° C., from about 20° C. to about 45° C., from about 25° C. to about 45° C., from about 30° C. to about 45° C. and from about 25° C. to about 40° C. In a preferred embodiment the temperature is about 28° C., 30° C. or 32° C. In a most preferred embodiment the temperature is about 28° C. or 30° C.

In some other embodiments, the fermentation is carried out for a period of time within the range of from about 8 hours to 240 hours, from about 8 hours to about 168 hours, from about 10 hours to about 144 hours, from about 15 hours to about 120 hours, or from about 20 hours to about 72 hours. Preferably the fermentation is carried out from about 20 hours to about 40 hours.

In some other embodiments, the fermentation is carried out at a pH in the range of about 4 to about 9, in the range of about 4.5 to about 8.5, in the range of about 5 to about 8, or in the range of about 5.5 to about 7.5. Preferably the fermentation will be carried out at a pH of 5-6.

In one embodiment of the method of producing ethyl acetate, the microorganism is cultured in a medium comprising between 1% and 30% (w/v) of a sugar, between 5% and 25% (w/v) of a sugar, between 10% and 20% (w/v) of a sugar, between 14% and 18% (w/v) of a sugar. Preferably the microorganism is cultured in a medium comprising between 15% and 20% (w/v) of a sugar.

A use of an ethyl acetate forming recombinant microorganism of the invention or a composition of the invention for the fermentative production of ethyl acetate is an additional embodiment of the invention. A further embodiment of the invention is a process for fermentative production of ethyl acetate comprising the steps of
I) growing the ethyl acetate forming microorganism of the invention in a fermenter and
II) recovering ethyl acetate from the fermentation broth obtained in I).

### Definitions

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine, prokaryotes also use the triplets “GTG” and “TTG” as startcodon. On the 3′-side it is bounded by one of the three triplets which specify stop codons (i.e., TAA, TAG, TGA). In addition a gene may include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Endogenous: An “endogenous” nucleotide sequence refers to a nucleotide sequence, which is present in the genome of a wild type microorganism.

Enhanced expression: “enhance” or “increase” the expression of a nucleic acid molecule in a microorganism are used equivalently herein and mean that the level of expression of a nucleic acid molecule in a microorganism is higher compared to a reference microorganism, for example a wild type. The terms “enhanced” or “increased” as used herein mean herein higher, preferably significantly higher expression of the nucleic acid molecule to be expressed. As used herein, an “enhancement” or “increase” of the level of an agent such as a protein, mRNA or RNA means that the level is increased relative to a substantially identical microorganism grown under substantially identical conditions. As used herein, “enhancement” or “increase” of the level of an agent, such as for example a preRNA, mRNA, rRNA, tRNA, expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a suitable reference microorganism. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, densitometric measurement of nucleic acid concentration in a gel, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a microorganism. Depending on the type of the induced protein product, its activity or the effect on the phenotype of the microorganism may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry OH et al. (1951) J Biol Chem 193:265-275) or measuring the absorption of CBB G-250 (Bradford M M (1976) Analyt Biochem 72:248-254).

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Foreign: The term “foreign” refers to any nucleic acid molecule (e.g., gene sequence) which is introduced into a cell by experimental manipulations and may include sequences found in that cell as long as the introduced sequence contains some modification (e.g., a point mutation, the presence of a selectable marker gene, etc.) and is therefore different relative to the naturally-occurring sequence.

Functional fragment: the term “functional fragment” refers to any nucleic acid and/or protein which comprises merely a part of the full length nucleic acid and/or full length polypeptide of the invention but still provides the same function, i.e. the function of an AAT enzyme catalyzing the reaction of acryloyl-CoA and butanol to n-BA and CoA. Preferably, the fragment comprises at least 50%, at least 60%, at least 70%, at least 80%, at least 90% at least 95%, at least 98%, at least 99% of the sequence from which it is derived. Preferably, the functional fragment comprises contiguous nucleic acids or amino acids of the nucleic acid and/or protein from which the functional fragment is derived. A functional fragment of a nucleic acid molecule encoding a protein means a fragment of the nucleic acid molecule encoding a functional fragment of the protein.

Functional linkage: The term “functional linkage” or “functionally linked” is equivalent to the term “operable linkage” or “operably linked” and is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form or can be inserted into the genome, for example by transformation.

Gene: The term “gene” refers to a region operably linked to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleoid but also the DNA of the self-replicating plasmid.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural genomic locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

Suitable hybridization conditions are for example hybridizing under conditions equivalent to hybridization in 7% sodium dodecyl sulfate (SDS), 0.5 M NaPO4, 1 mM EDTA at 50° C. with washing in 2×SSC, 0.1% SDS at 50° C. (low stringency) to a nucleic acid molecule comprising at least 50, preferably at least 100, more preferably at least 150, even more preferably at least 200, most preferably at least 250 consecutive nucleotides of the complement of a sequence. Other suitable hybridizing conditions are hybridization in 7% sodium dodecyl sulfate (SDS), 0.5 M NaPO4, 1 mM EDTA at 50° C. with washing in 1×SSC, 0.1% SDS at 50° C. (medium stringency) or 65° C. (high stringency) to a nucleic acid molecule comprising at least 50, preferably at least 100, more preferably at least 150, even more preferably at least 200, most preferably at least 250 consecutive nucleotides of a complement of a sequence. Other suitable hybridization conditions are hybridization in 7% sodium dodecyl sulfate (SDS), 0.5 M NaPO4, 1 mM EDTA at 50° C. with washing in 0.1×SSC, 0.1% SDS at 65° C. (very high stringency) to a nucleic acid molecule comprising at least 50, preferably at least 100, more preferably at least 150, even more preferably at least 200, most preferably at least 250 consecutive nucleotides of a complement of a sequence.

“Identity”: “Identity” when used in respect to the comparison of two or more nucleic acid or amino acid molecules means that the sequences of said molecules share a certain degree of sequence similarity, the sequences being partially identical. To determine the percentage identity (homology is herein used interchangeably if referring to nucleic acid sequences) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are identical at this position. The percentage identity between the two sequences is a function of the number of identical positions shared by the sequences (i.e. % identity=number of identical positions/total number of positions×100). The terms “homology” and “identity” are thus to be considered as synonyms when referring to nucleic acid sequences. When referring to amino acid sequences the term identity refers to identical amino acids at a specific position in a sequence, the term homology refers to homologous amino acids at a specific position in a sequence. Homologous amino acids are amino acids having a similar side chain. Families of amino acid residues having similar side chains have been defined in the art.

A nucleic acid molecule encoding a protein homologous to a protein of the invention can be created by introducing one or more nucleotide substitutions, additions or deletions into a nucleotide sequence such that one or more amino acid substitutions, additions or deletions are introduced into the encoded protein. Mutations can be introduced into one of the sequences of the invention by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein of the invention is preferably replaced with another amino acid residue from the same side chain family.

Alternatively, in another embodiment, mutations can be introduced randomly along all or part of a coding sequence, such as by saturation mutagenesis, and the resultant mutants can be screened for the respective activity described herein to identify mutants that retain their activity. Following mutagenesis of one of the sequences of the invention, the encoded protein can be expressed recombinantly and the activity of the protein can be determined using, for example, assays described herein.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444(1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparisons of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wisconsin, USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence identity are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has a 80% identity. Preferably the identity is calculated on the complete length of the query sequence, for example SEQ ID NO:1.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring nucleic acid molecule or polypeptide present in a living cell is not isolated, but the same nucleic acid molecule or polypeptide, separated from some or all of the coexisting materials in the natural system, is isolated. Such nucleic acid molecules can be part of a vector and/or such nucleic acid molecules or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a genomic or plasmid location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Non-coding: The term “non-coding” refers to sequences of nucleic acid molecules that do not encode part or all of an expressed protein. Non-coding sequences include but are not limited enhancers, promoter regions, 3′ untranslated regions, and 5′ untranslated regions.

Nucleic acids and nucleotides: The terms “nucleic acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “nucleic acid molecule”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Promoter: The terms “promoter”, or “promoter sequence” are equivalents and as used herein, refer to a DNA sequence which when operably linked to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. The promoter does not comprise coding regions or 5′ untranslated regions. The promoter may for example be heterologous or homologous to the respective cell. A nucleic acid molecule sequence is “heterologous to” an organism or a second nucleic acid molecule sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host.

Purified: As used herein, the term “purified” refers to molecules, either nucleic or amino acid sequences that are removed from their natural environment, isolated or separated.

“Substantially purified” molecules are at least 60% free, preferably at least 75% free, and more preferably at least 90% free from other components with which they are naturally associated. A purified nucleic acid sequence may be an isolated nucleic acid sequence.

Significant increase: An increase for example in enzymatic activity, gene expression, productivity or yield of a certain product, that is larger than the margin of error inherent in the measurement technique, preferably an increase by about 10% or 25% preferably by 50% or 75%, more preferably 2-fold or-5 fold or greater of the activity, expression, productivity or yield of the control enzyme or expression in the control cell, productivity or yield of the control cell, even more preferably an increase by about 10-fold or greater.

Significant decrease: A decrease for example in enzymatic activity, gene expression, productivity or yield of a certain product, that is larger than the margin of error inherent in the measurement technique, preferably a decrease by at least about 5% or 10%, preferably by at least about 20% or 25%, more preferably by at least about 50% or 75%, even more preferably by at least about 80% or 85%, most preferably by at least about 90%, 95%, 97%, 98% or 99%.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with at least one recombinant nucleic acid molecule.

Vector: As used herein, the term “vector” refers to a nucleic acid molecule capable of transporting another nucleic acid molecule to which it has been linked. One type of vector is a genomic integrated vector, or “integrated vector”, which can become integrated into the genomic DNA of the host cell. Another type of vector is an episomal vector, i.e., a plasmid or a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

Wild type: The term “wild type”, “natural” or “natural origin” means with respect to an organism that said organism is not changed, mutated, or otherwise manipulated by man. With respect to a polypeptide or nucleic acid sequence, that the polypeptide or nucleic acid sequence is naturally occurring or available in at least one naturally occurring organism which is not changed, mutated, or otherwise manipulated by man.

A wild type of a microorganism refers to a microorganism whose genome is present in a state as before the introduction of a genetic modification of a certain gene. The genetic modification may be e.g. a deletion of a gene or a part thereof or a point mutation or the introduction of a gene.

The terms “production” or “productivity” are art-recognized and include the concentration of the fermentation product (for example, dsRNA) formed within a given time and a given fermentation volume (e.g., kg product per hour per liter). The term “efficiency of production” includes the time required for a particular level of production to be achieved (for example, how long it takes for the cell to attain a particular rate of output of a fine chemical).

The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules or of useful recovered molecules of that compound in a given amount of culture over a given amount of time is increased.

The term “recombinant microorganism” includes microorganisms which have been genetically modified such that they exhibit an altered or different genotype and/or phenotype (e. g., when the genetic modification affects coding nucleic acid sequences of the microorganism) as compared to the wild type microorganism from which it was derived. A recombinant microorganism comprises at least one recombinant nucleic acid molecule.

The term “recombinant” with respect to nucleic acid molecules refers to nucleic acid molecules produced by man using recombinant nucleic acid techniques. The term comprises nucleic acid molecules which as such do not exist in nature or do not exist in the organism from which the nucleic acid molecule is derived, but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecules” may also comprise a “recombinant construct” which comprises, preferably operably linked, a sequence of nucleic acid molecules not naturally occurring in that order. Preferred methods for producing said recombinant nucleic acid molecules may comprise cloning techniques, directed or non-directed mutagenesis, gene synthesis or recombination techniques.

An example of such a recombinant nucleic acid molecule is a plasmid into which a heterologous DNA-sequence has been inserted or a gene or promoter which has been mutated compared to the gene or promoter from which the recombinant nucleic acid molecule derived. The mutation may be introduced by means of directed mutagenesis technologies known in the art or by random mutagenesis technologies such as chemical, UV light or x-ray mutagenesis or directed evolution technologies.

The term “directed evolution” is used synonymously with the term “metabolic evolution” herein and involves applying a selection pressure that favors the growth of mutants with the traits of interest. The selection pressure can be based on different culture conditions, ATP and growth coupled selection and redox related selection. The selection pressure can be carried out with batch fermentation with serial transferring inoculation or continuous culture with the same pressure.

The term “expression” or “gene expression” means the transcription of a specific gene(s) or specific genetic vector construct. The term “expression” or “gene expression” in particular means the transcription of gene(s) or genetic vector construct into mRNA. The process includes transcription of DNA and may include processing of the resulting RNA-product. The term “expression” or “gene expression” may also include the translation of the mRNA and therewith the synthesis of the encoded protein, i.e. protein expression.

### EXAMPLES

The following examples serve to illustrate the invention without limiting the scope thereof.

### Chemicals and Common Methods

Unless indicated otherwise, cloning procedures carried out for the purposes of the present invention including restriction digest, agarose gel electrophoresis, purification of nucleic acids, ligation of nucleic acids, transformation, selection and cultivation of bacterial cells are performed as described (Sambrook et al., 1989). Sequence analyses of recombinant DNA are performed with a laser fluorescence DNA sequencer (Applied Biosystems, Foster City, CA, USA) using the Sanger technology (Sanger et al., 1977). Unless described otherwise, chemicals and reagents are obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases are from New England Biolabs (Ipswich, MA, USA) or Roche Diagnostics GmbH (Penzberg, Germany). Oligonucleotides are synthesized by IDT (Coralville, USA).

### 1. In Vivo Production of Acryloyl-CoA in S. cerevisiae

### 1.1 Heterologous Expression of Short-Chain Acyl-CoA Oxidase (Aco) in S. cerevisiae

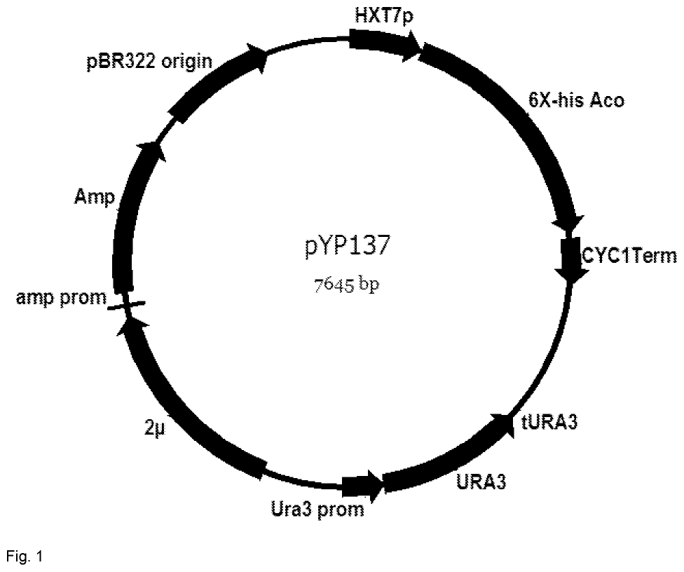

Short-chain acyl-CoA (coenzyme A) oxidase catalyses an oxidation reaction with saturated acyl-CoAs (e.g. propionyl-CoA) to enoyl-CoAs (e.g. acryloyl-CoA). Nucleotide sequence of the Aco gene (GB: AB017643.1) from Arabidopsis thaliana was obtained from the NCBI (http://www.ncbi.nlm.nih.gov/). The nucleotide sequence was codon optimized for expression in yeast with an N-terminal 6x-His tag based on the standard codon usage table in IDT Gene synthesis service (Seq ID No. 57). The 1337 bp of ACO gene was synthesized by IDT (Coralville, USA). The ACO gene fragment flanked by BamHI and HindIII restriction sites was inserted in a vector with 2 micron and pBR322 origin of replicon, ura3 and bla gene as markers to yield pYP137 (high-copy E. coli/S. cerevisiae shuttle vector; complements Ura-auxotrophy in S. cerevisiae: pBR322; CEN4-origin; AmpR; URA3, ACO under control of truncated HXT71-392 promoter and CYC1 terminator, Seq ID No. 67). The construct is subjected to be introduced in S. cerevisiae with various combinations of other genes in the pathway (FIG. 1).

### 1.2 Heterologous Expression of Propionyl-CoA Transferase in S. cerevisiae

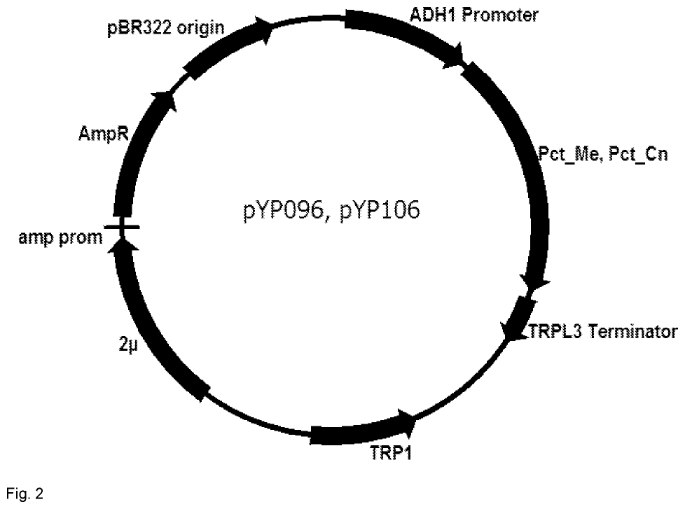

Enzymatic activity of pct is to transfer CoA to short carbon length acids by forming thioester bond. This yield various acyl-CoAs as intermediates of various biosynthetic pathways. Propionyl-CoA transferases use acetyl-CoA as a CoA donor to create propionyl-CoA from propionate. Pct genes used for this experiment from Megasphaera elsdenii, (CCC72964) and from Cupriavidus necator (CAJ93797) were codon-optimized for expression in S. cerervisiae and synthesized by GeneScript (Piscataway, USA) and IDT (Coralville, USA), yielding pct-Me (Seq ID No. 59) and pct-CN (Seq ID No. 61) (Prabhu et. al 2012). These genes were cloned into gene overexpression plasmids by homologous recombination methods described in prior publications (Gietz et. al., 2007). The pct genes were inserted seamlessly into the E. coli/S. cerevisiae shuttle plasmid to overexpress the protein from the strong constitutive promoter ADH1 and TRPL3 terminator with tryptophan auxotrophic marker in 2 micron base high-copy expression vector. The resulting plasmids pYP096 (Seq ID No. 65), and pYP106 (Seq ID No. 66) are listed in the Table 1 (FIG. 2).

TABLE 1
List of plasmids encoding for different propionate CoA-transferase

| | | Enzyme | | Accession #, |
|---|---|---|---|---|
| Plasmid | Plasmid description | Key | Species/Protein | Seq ID No. |
| pYP096 | high-copy E. coli/S. cerevisiae | Pct-Me | Megasphaera | CCC72964, |
| | shuttle vector; complements | | elsdenii | Seq ID No. 65 |
| | Trp-auxotrophy in S. cerevisiae: | | | |
| | pBR322; 2 μm-ori; AmpR; TRP1; | | | |
| | ADH1 promoter and RPL3 terminator, | | | |
| | contains codon-optimized pct of | | | |
| | M. elsdenii | | | |
| pYP106 | high-copy E. coli/S. cerevisiae | Pct-Cn | Cupriavidus | CAJ93797, |
| | shuttle vector; complements | | necator | Seq ID No. 66 |
| | Trp-auxotrophy in S. cerevisiae: | | | |
| | pBR322; 2 μm-ori; AmpR; TRP1; | | | |
| | ADH1 promoter and RPL3 terminator, | | | |
| | contains codon-optimized pct of | | | |
| | Cupriavidus necator | | | |

### 2. In Vivo Production of Lactoyl-CoA in S. cerevisiae

In order to produce lactoyl-CoA in the cytosol of S. cerevisiae, conversion of pyruvate to lactate and lactate to lactoyl-CoA has to occur by heterologous enzymes. Lactate dehydrogenase is responsible to convert pyruvate to lactate with NADH, and lactate dehydrogenase (IdhA) from E. coli was expresses in yeast after codon optimization, yielding IdhA-sc (Seq ID No. 69). To overexpress IdhA-sc in S. cerevisiae, a yeast shuttle expression vector was constructed. The IdhA-sc was inserted seamlessly by homologues recombination between the HXT7 promoter and CYC1 terminator on a plasmid (high-copy E. coli/S. cerevisiae shuttle vector; complements His-auxotrophy in S. cerevisiae: pBR322; 2 μ-ori; AmpR; HIS3). The resulting plasmid was named pYP024 (Seq ID No. 71). This plasmid was transformed into S. cerevisiae W303-1A strain to yield strain TYC-006. TYC-006 was cultured in the synthetic media and the cultured broth contained lactate which was converted from lactate. This lactate was converted further to lactoyl-CoA by expression of propionyl-CoA transferase, pct-Me (Seq ID No. 59), and pct-Cn (Seq ID No. 61) in the S. cerevisiae strain with IdhA-sc.

### 3. Heterologous Expression of Alcohol Acyl Transferase (AAT) in P. pastoris and S. cerevisiae

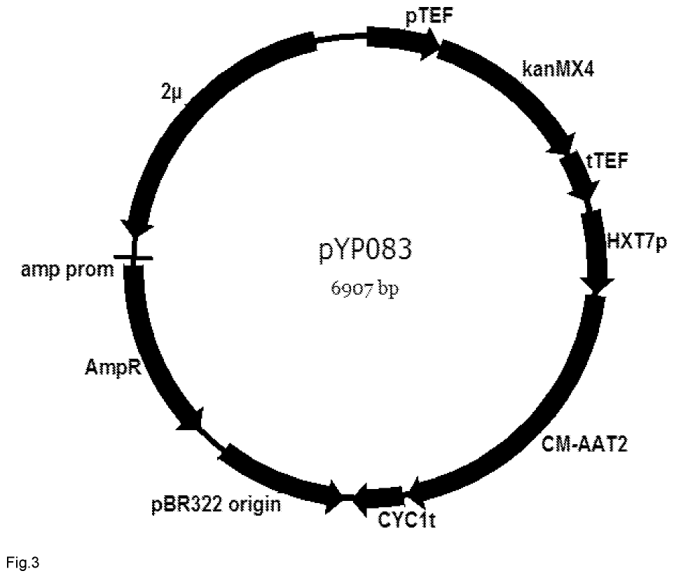

AAT is responsible for forming an ester bond between alcohols and acyl-CoAs. Various putative AATs were chosen for analysis (see Table 2). Selected AAT genes were subjected to go through activity screening tests in vitro. The methylotrophic yeast P. pastoris was chosen as expression host for the evaluation of expression of candidate genes encoding putative AATs. Expression constructs were set up in the plasmid pD902 (DNA 2.0) which provides the strong methanol-inducible AOX promoter. The plasmid was modified by inserting the PARS1 element, which allowed the episomal replication of the plasmid (Cregg, J. M., et al., 1985). Selected candidate AATs were constructed into pD902e plasmids (Seq ID No. 68) and transformed in P. pastoris GapChap. In order to overexpress for example Cm-AAT2 in S. cerevisiae, the gene was cloned in a high copy overexpression vector with a strong promoter and a terminator, yielding pYP083 (Seq ID No. 64, high-copy E. coli/S. cerevisiae shuttle vector; confers geneticin resistance: pBR322; 2 μm-ori; AmpR; lac Z; kanMX; truncated HXT71-392 promoter and CYC1 terminator CDS: N-terminal His tagged CmAAT2_Cucumis melo) (FIG. 3).

TABLE 2
Overview of the AATs selected for expression and characterization

| SEQ | | | | | |
|---|---|---|---|---|---|
| ID | gene | | | | |
| NO | name | identifier | Origin | Origin | reference |
| 1 | CM-AAT1 | CAA94432 | Cucumis | melon | Yahyaoui et |
| | | | melo | | al., 2002 El-Sharka- |
| | | | | | way et al., 2005 |
| 3 | MpAAT1 | AY707098 | Malus | apple | Souleyre et al., 2005 |
| | | | pumila | | |
| 5 | VAAT | CAC09062 | Fragaria | strawberry | Beckwilder et |
| | | | vesca | | al., 2004 |
| 7 | CM-AAT2 | AAL77060 | Cucumis | melon | El-Sharkaway et |
| | | | melo | | al., 2005 |
| 9 | Md-AAT2 | AAS79797 | Malus | apple | Li et al., 2006 |
| | | | domestica | | |
| 11 | BEBT | AF500200 | Clarkia | flower | D'Auria et al., 2002 |
| | | | breweri | | |
| 13 | CbBEAT | AAC18062 | Clarkia | flower | Dudareva et |
| | | | breweri | | al., 1998 |
| 15 | SAAT | CAC09048 | Fragaria × | strawberry | Beekwilder et al., |
| | | | ananassa | | 2004 |
| 17 | FaAAT2 | JN089766) | Fragaria × | strawberry | Cumplido-Laso et |
| | | | ananassa | | al., 2012 |
| 19 | AeAT9 | HO772637 | Actinidia | kiwi | Günther et al., 2011 |
| | | | eriantha | | |
| 21 | Rh-AAT1 | AAW31948 | Rosa hybrid | flower | Guterman et |
| | | | cultivar | | al., 2006 |
| 23 | CM-AAT4 | AAW51126 | Cucumis | melon | El-Sharkaway et |
| | | | melo | | al., 2005 |
| 25 | ACT | WP_001010387 | (Staphylococcus | bacterium | Rodriguez et |
| | | | sciuri) | | al., 2014 |
| 27 | BanAAT | AX025506 | Musa | banana | Beekwilder et |
| | | | sapientum | | al., 2004 |
| 29 | Glossy2 | CAA61258 | Zea mays | maize | Tacke et al., 1995 |
| 31 | CM-AAT3 | AAW51125 | Cucumis | melon | El-Sharkaway et |
| | | | melo | | al., 2005 |
| 33 | BAHDFox | EMT69722 | Fusarium | fungi | — |
| | | | oxysporum | | |
| 35 | VpAAT1 | FJ548611 | Vasconcellea | papaya | Balbotin et al., 2010 |
| | | | pubescens | | |
| 37 | AMAT | AY705388 | Vitis | grape | Wang & De |
| | | | labrusca | | Luca, 2005 |
| 39 | Pun1 | AAV66311 | Capsicum | pepper | Stewart et al., 2005 |
| | | | annum | | |
| 41 | Dv3MaT | AAO12206 | Dahlia | flower | Suzuki et al., 2002 |
| | | | variabilis | | |
| 43 | NtHCT | CAD47830 | Nicotiana | tobacco | Hoffmann et |
| | | | tababcum | | al., 2003 |
| 45 | DBATAca | ACI47063 | Aspergillus | fungi | — |
| | | | candidus | | |
| 47 | TSga | WP_006129805 | Streptomyces | bacterium | — |
| | | | gancidicus | | |
| 49 | TSvi | YP_004810992 | Streptomyces | bacterium | — |
| | | | violaceusniger | | |
| 51 | CAT | YP_007500975 | Shigella | bacterium | Rodriguez et |
| | | | sonnei | | al., 2014 |
| 53 | EHT | NP_009736 | Saccharomyces | yeast | Rodriguez et |
| | | | cerevisiae | | al., 2014 |
| 55 | ATF | NP_015022 | Saccharomyces | yeast | Rodriguez et |
| | | | cerevisiae | | al., 2014 |

### 4. In Vivo Production of Butanol in S. cerevisiae

A butanol producing S. cerevisiae (TYC-185) strain was established as described in Schadeweg, V. and E. Boles, 2016.

### 5. In Vivo Production of nBA by Feeding Substrate

We have established a pathway within S. cerevisiae that is able to create n-butylacrylate from feeding of propionate and n-butanol. The first step uses propionyl-CoA transferase (M. elsdenii) to convert propionate to propionyl-CoA. Then, the Acyl-CoA dehydrogenase (ACO) enzyme from A. thaliana to convert the propionyl-CoA to acryloyl-CoA. Further down the pathway, acryloyl-CoA and n-butanol become key intermediates, which are esterified by the activity of an alcohol acyltransferase (AAT) to the desired end product n-butylacrylate. We used TYC-072 modified strain of S. cerevisiae to introduce nBA biosynthetic pathway plasmids. TYC-072 was transformed with a set of plasmids, pYP137 (Seq ID No. 67), pYP096 (Seq ID No. 65), and pYP083 (Seq ID No. 64) from which Aco, pct-Me, and cm-AAT2 are overexpressed, to yield S. cerevisiae strain TYC-166. TYC-166 was cultured in synthetic defined SD media (Bacto-Yeast nitrogen base without amino acids, 1.7 g; Glucose, 20 g; Dropout mix, 2 g/1 L) with G418 and without TRP and URA. As a negative control, an empty control vector instead of the Aco vector was introduced into TYC-072 with pct-Me and cm-AAT2 overexpression vectors to yield S. cerevisiae strain TYC-181. These strains were grown in SE-TRP-URA+G418 selective minimal media (glutamic acid, 1 g; Bacto-Yeast nitrogen base without amino acids and ammonium sulfate, 1.7 g; Dropoutmix, 2 g; glucose 20 g/1 L) at 30° C. (Table 3).

TABLE 3
Strains to produce n-butylacrylate in S. cerevisiae.

| Strain | | | Selection |
|---|---|---|---|
| Name | Description of strain | plasmids | markers |
| TYC-72 | MATa; ura3-52; trp1-289; leu2-3_112; | none | Auxotrophic: |
| | his3 Δ1; MAL2-8C; SUC2 adh1::loxP | | Trp, Ura, |
| | adh3::loxP; adh4Δ::loxP, adh5Δ::loxP | | Leu, His |
| | Δadh1,3,4,5 (all with loxP), Ethanol non- | | |
| | producer | | |
| TYC-166 | Prepared from TYC-072, Ethanol | pYP083 (Cm-AAT2) | Dominant: G418 |
| | non-producer, overexpress Aco, pct-Me, | pYP096 (pct-ME) | Auxotrophic: |
| | and Cm-AAT2 | pYP137 (ACO) | Trp, Ura |
| TYC-181 | Prepared from TYC-072, Ethanol non- | pYP004 (empty) | Dominant: |
| | producer, overexpress pct-Me, and Cm- | pYP083 (Cm- | G418 |
| | AAT2 | AAT2) | Auxotrophic: |
| | | pYP096 (pct-ME) | Trp, Ura |
| TYC-185 | n-Butanol producer. Ethanol non- | none | Auxotrophic: |
| | producer, MATa; ura3-52; trp1-289; | | Ura, Trp, His |
| | leu2 3_112; his3Δ1; MAL2-8C; SUC2; | | |
| | adh1::loxP; adh2Δ::LEU2; adh3::loxP; | | |
| | adh4Δ::loxP; adh5::loxP; adh6Δ::coaA, | | |
| | natNT2; | | |
| | sfa1Δ::adhE, A267T/E568K, hphNT1; | | |
| | gpd2::ERG10, hbd, crt, ter, adhE2, EutE, | | |
| | KanMX | | |

Strains were grown aerobically in test tubes from glycerol stocks in 10 mL of SE-TRP-URA+G418 minimal media overnight at 30 ° C. and 250 rpm. These cultures were then transferred into a 250 mL baffled glass shake flask and normalized to an OD600 of 0.2 for a 25 mL culture. 3.0 g/L Sodium Propionate and 0.5% butanol were fed to the cultures every 24 hours. An additional 2% of glucose was also fed after the first 24 hours and every 24 hours thereafter. Samples were taken at 3, 6, 9, 12, 24, 36 and 48 hour time points for HPLC and Solid Phase Micro Extraction (SPME) detection. The SPME method was used to detect esters, specifically Butylacrylate, and Butyl propionate.

### 6. In Vivo Production of nBA from Glucose

In order to demonstrate nBA production in microorganism from glucose as a carbon source, multiple pathways are introduced to generate substrates to the final esterification step, which is performed by AAT enzymes. The two major pathways to produce two key intermediates are heterologous biosynthetic pathways for butanol and acryloyl-CoA. Two S. cerevisiae production host as described in the examples 1 and 4 showed production of acryloyl-CoA and butanol in separate experiments. We use the S. cerevisiae strain (TYC-185), which can produce butanol by reverse beta-oxidation described in example 4 as a base strain to add an acryloyl-CoA pathway and alcohol acyl transferase (AAT). AAT and genes for the acryloyl-CoA pathway, short chain acyl-CoA oxidase (ACO), propionyl-CoA transferase (pct-Me), methylmalonyl-CoA mutase, methylmalonyl-CoA decarboxylase, are integrated into the chromosome of the base strain with functional promoters and terminators. In addition, other acryloyl-CoA production pathways are used separately and/or collectively. Some of example of other acryloyl-CoA pathways are the lactate route and the 3HP route. The lactate route is composed of a set of enzymes to convert pyruvate to lactate and from lactate to lactoyl-CoA and then to acryloyl-CoA. A 3HP route is composed of a set of enzymes to convert malonyl-CoA or beta-alanine to 3-oxopropanate, which is converted to 3-hydroxypropanoate (3HP) and further to 3-hydroxypropanoyl-CoA and then form acryloyl-CoA. Other routes from glucose to 3HP to acryloyl-CoA are tested. Optimization of the protein expression is achieved by testing various promoters, integration loci, copy-number of genes, episomal plasmid expression, and culture conditions. Once all the necessary genes are expressed in S. cerevisiae, glucose is converted to acryloyl-CoA and together with butanol then esterified by an AAT to form nBA and/or other ester compounds.

### 7. Production of Other Ester Compounds

### 7.1 In Vivo Production of Other Esters

Various ester compound were produced in the engineered S. cerevisiae strains with expression of heterologous pathways for nBA formation. TYC-166 and TYC-181 strains, in which an AAT and pct gene were overexpressed, showed production of n-butylpropionate (nBP) as a by-product along with nBA production. Propionate was fed to the culture broth and transformed by the cells to propionyl-CoA due to the enzyme activity of pct-ME. The propionyl-CoA together with fed butanol were esterified by the AAT activity resulting in production of n-butyl propionate (nBP). Detailed experimental methods are described in example 5. nBP in the culture broth was detected by the methods described in Example 8. Additionally, in vivo production of butyllactate was demonstrated by expression of lactate dehydrogenase and AAT in yeast.

### 7.2 In Vitro Production of nBA and Other Esters by AATs

In addition to nBA, in vitro formation of other ester compounds, such as butyl propionate, butyl lactate, butyl acetate, and ethyl acetate, were confirmed by in vitro enzyme activity assays using the activity of the purified AAT enzymes, which form ester compounds from acyl-CoAs and alcohols. The methylotrophic yeast P. pastoris GapChap, which provides a chaperonin co-expression, was chosen as expression host for the evaluation of expression of candidate genes encoding putative AATs. Plasmid constructs with N-terminal 6X-his-tagged AATs were cloned with the strong methanol-inducible AOX promoter. The cultures of individual constructs were pooled, washed once with 100 mM sodium phosphate buffer pH 7.5 and re-suspended in 50 mM sodium phosphate buffer pH 8.0 containing complete plus EDTA free protease inhibitor (Roche), 300 mM NaCl, mM imidazole. A Branson Sonifier 250 was used to generate a crude cell extract; 8×5 min pulses with 50% duty cycle and output level 7 were used to disrupt the cells in an appropriate vessel on ice. After centrifugation the supernatant was filtered before loading onto a HisTrap HF Ni-NTA column (1 ml, GE Healthcare) equilibrated with buffer A (300 mM NaCl, 50 mM sodium phosphate buffer pH 8.0, 10 mM imidazole). Buffer A was also used for loading and washing. A gradient was applied by switching from buffer A to buffer B (300 mM NaCl, 50 mM sodium phosphate buffer pH 8.0, 500 mM imidazole) within 10 column volumes (CV). Elution was prolonged by 5 additional CV of buffer B. Fractions of 1 ml were collected and separately analyzed by SDS-PAGE and activity measurements (GC/MS) for the identification of AAT protein. Up to 3 fractions were pooled and desalted by size exclusion chromatography (PD10, GE-Healthcare). Final preparations contained 100 mM sodium phosphate buffer pH 7.5 and were stored on ice. Purity was analyzed by SDS-PAGE and densitometric analysis of the corresponding protein band. Total protein amount was determined by Micro-BCA Assay (Thermo Fisher). The assay to determine the activity of AATs was set up as follows: 100 mM potassium phosphate buffer pH 7.5, 5 mM alcohol (e.g. butanol), 0.5 mM acyl-CoA, 1 mg/mL BSA, and 20 μl enzyme sample in a total volume of 100 μl in a 2 mL glass vial, which was sealed immediately after setup. Samples were set up in duplicate and incubated at room temperature (RT) for 0, 2, 4, 8 and 24 h respectively. Subsequently enzymes were inactivated by heat denaturation at 65° C. for 20 min. Afterward samples were analyzed by the methods described in Example 8. Various AATs showed esterase activities to various substrates to form butylacrylate, butyl propionate, butyl lactate, butyl acetate, and ethyl acetate. (Table. 4)

TABLE 4
Activities of AATs towards the formation of variable compounds.

| SEQ ID | | butyl | butyl | butyl | ethyl |
|---|---|---|---|---|---|
| NO | Enzyme | acrylate | propionate | lactate | acetate |
| 2 | Cm-AAT1 | ∘ | ∘ | ∘ | x |
| 4 | Mp-AAT1 | ∘ | ∘ | ∘ | ∘ |
| 6 | VAAT | ∘ | ∘ | ∘ | ∘ |
| 8 | CM-AAT2 | ∘ | ∘ | ∘ | ∘ |
| 10 | Md-AAT2 | ∘ | ∘ | ∘ | ∘ |
| 12 | BEBT | ∘ | ∘ | x | ∘ |
| 14 | CbBEAT | ∘ | ∘ | x | ∘ |
| 16 | SAAT | x | ∘ | ∘ | ∘ |
| 18 | Fa-AAT2 | x | ∘ | ∘ | ∘ |
| 20 | AeAT9 | x | ∘ | ∘ | ∘ |
| 22 | Rh-AAT1 | x | ∘ | x | ∘ |
| 24 | CM-AAT4 | x | ∘ | x | ∘ |
| 26 | ACT | x | ∘ | x | ∘ |
| 28 | BanAAT | x | x | x | ∘ |

∘: detected,
x: not detected

### 8. Detection of nBA from Culture Broth

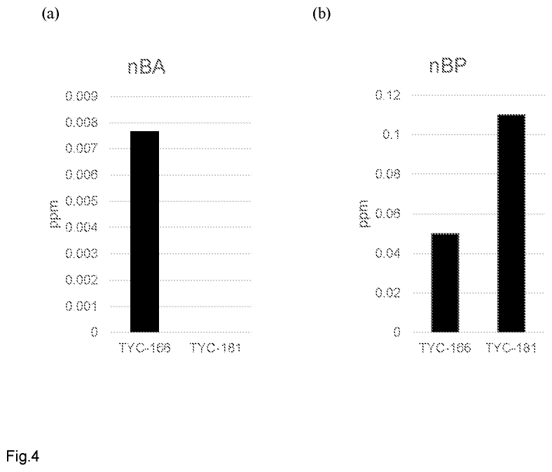

Solid phase micro extraction (SPME)/GC/MS was used to detect nBA and other ester compounds from the culture broth. SPME samples were prepared by adding 500 μL of filtered (0.22 μm) cultured media into the head space analysis vial. SPME was done with carboxen/polydimethylsiloxane fiber. Extraction was done at 40° C. for 15 min after samples were conditioned at 40° C. for 10 min. Desorption was carried out at injection port at 250° C., followed by GC separation (column DB-624) and MS detection (full scan mode). nBA was detected from the broth of TYC-166 culture. No nBA was detected from TYC-181, which was negative control experiment. Both strains produced n-butylpropionate as a by-product formed by esterification of butanol and propionyl-CoA (FIG. 4).

What is claimed is:

1. A recombinant microorganism comprising a nucleic acid molecule encoding an AAT gene encoding an AAT enzyme having an n-butylacrylate (n-BA) forming activity, wherein the microorganism also comprises a butanol producing pathway and an acryloyl-CoA producing pathway, and wherein the microorganism has an introduced, increased, or enhanced n-butylacrylate forming activity and/or expression of said AAT enzyme;
wherein the AAT gene encoding an AAT enzyme having an n-BA forming activity is selected from the group consisting of:
(I) a nucleic acid molecule comprising a sequence selected from the group consisting of SEQ ID NO: 1, 3, 5, 7, 9, 11 and 13;
(II) a nucleic acid molecule having at least 80% identity to a nucleic acid molecule selected from the group consisting of SEQ ID NO: 1, 3, 5, 7, 9, 11 and 13;
(III) a nucleic acid molecule hybridizing to the complement of a nucleic acid molecule having SEQ ID NO: 1, 5, 7, 9, 11 and 13;
(IV) a nucleic acid molecule encoding a polypeptide selected from the group consisting of SEQ ID NO: 2, 6, 8, 10, 12, and 14; and
(V) a nucleic acid molecule encoding a polypeptide having at least 60% identity to a polypeptide selected from the group consisting of SEQ ID NO: 2, 6, 8, 10, 12, and 14.

2. The recombinant microorganism of claim 1, wherein the microorganism is selected from a genus of the group consisting of Saccharomyces, Yarrowia, Arxula, Kluyveromyces, Lactobacillus, Clostridium, Pseudomonas, Corynebacterium, Bacillus, Erwinia, Escherichia, Pantoea, Streptomyces, Zymomonas and Rhodococcus.

3. A composition comprising one or more recombinant microorganisms according to claim 1.

4. The composition of claim 3 further comprising a medium and a carbon source.

5. A method for producing a recombinant microorganism producing n-BA comprising the steps of:
(I) introducing, increasing, or enhancing the activity and/or expression of an AAT gene encoding an AAT enzyme having an n-BA forming activity in a microorganism; and
(II) further introducing in the microorganism a butanol producing pathway and an acryloyl-CoA producing pathway;
wherein the AAT gene encoding an AAT enzyme having an n-BA forming activity is selected from the group consisting of:
(I) a nucleic acid molecule comprising a sequence selected from the group consisting of SEQ ID NO: 1, 3, 5, 7, 9, 11 and 13;
(II) a nucleic acid molecule having at least 80% identity to a nucleic acid molecule selected from the group consisting of SEQ ID NO: 1, 3, 5, 7, 9, 11 and 13;
(III) a nucleic acid molecule hybridizing to the complement of a nucleic acid molecule having SEQ ID NO: 1, 5, 7, 9, 11 and 13;
(IV) a nucleic acid molecule encoding a polypeptide selected from the group consisting of SEQ ID NO: 2, 6, 8, 10, 12, and 14; and
(V) a nucleic acid molecule encoding a polypeptide having at least 60% identity to a polypeptide selected from the group consisting of SEQ ID NO: 2, 6, 8, 10, 12, and 14.

6. The method of claim 5, wherein the microorganism is selected from a genus of the group consisting of Saccharomyces, Lactobacillus, Clostridium, Pseudomonas, Corynebacterium, Bacillus, Erwinia, Escherichia, Pantoea, Streptomyces, Zymomonas and Rhodococcus.

7. A recombinant expression construct comprising a promoter functional in a microorganism functionally linked to a nucleic acid molecule having a sequence selected from the group consisting of:
(I) a nucleic acid molecule comprising a sequence of SEQ ID NO: 1, 3, 5, 7, 9, 11 or 13; and
(II) a nucleic acid molecule comprising a sequence of SEQ ID NO: 1, 3, 5, 7, 9, 11 or 13; and
(III) a nucleic acid molecule hybridizing to the complement of a nucleic acid molecule having SEQ ID NO: 1, 5, 7, 9, 11 or 13 under stringent conditions; and
(IV) a nucleic acid molecule encoding a polypeptide of SEQ ID NO: 2, 6, 8, 10, 12, or 14, or a functional fragment thereof; and
(V) a nucleic acid molecule encoding a polypeptide having at least 60% identity to a polypeptide of SEQ ID NO: 2, 6, 8, 10, 12, or 14, or a functional fragment thereof,
wherein the promoter is heterologous to the nucleic acid molecule.

8. A recombinant vector comprising the recombinant expression construct of claim 7.

9. A method of culturing or growing a recombinant microorganism comprising inoculating a culture medium with one or more recombinant microorganism according to claim 1 and culturing or growing the one or more recombinant microorganism in the culture medium.


SEQUENCE LISTING (71 sequences)

SEQ ID NO: 1 - DNA, 1407 bp, Cucumis melo
  misc_feature 1..1407: note = CM-AAT1
  source 1..1407: mol_type = genomic DNA
atgcatcatcaccaccaccacgagactatgcagactatcgatttctcattccacgttagaaagtgtcagccagagttgatcgctccagctaacccaactccatacgagttcaagcaattgtccgacgttgacgaccaacagtccttgagattgcagttgccattcgttaacatctacccacacaacccatccttggagggtagagatccagttaaggttatcaaagaggctatcggtaaggctttggttttctactacccattggctggtagattgagagagggtccaggtagaaagttgttcgttgagtgtactggtgagggtatcttgttcattgaagctgacgctgacgtttccttggaagagttctgggatactttgccatactccttgtcctccatgcagaacaacatcatccacaacgctttgaactccgacgaggttttgaactcccctttgttgttgatccaggttactagattgaagtgtggtggtttcatcttcggtttgtgtttcaaccacactatggctgacggtttcggtatcgttcagttcatgaaggctactgctgagatcgctagaggtgctttcgctccatctattttgccagtttggcagagagctttgttgactgctagagatccaccaagaatcactttcagacactacgagtacgaccaggttgttgacatgaagtccggtttgatcccagttaactccaagatcgaccagttgttcttcttctcccaattgcaaatctccactttgagacagactttgccagctcacttgcacgactgtccatctttcgaagttttgactgcttacgtttggagattgagaactatcgctttgcagttcaagccagaggaagaggttagattcctttgtgttatgaacttgagatccaagattgacatcccattgggttactacggtaacgctgttgttgttccagctgttatcactactgctgctaagttgtgtggtaaccctttgggttacgctgttgacttgatcagaaaggctaaggctaaagctactatggaatacatcaagtccactgttgatttgatggttatcaagggtagaccatacttcactgttgttggttccttcatgatgtccgacttgactagaatcggtgttgagaacgttgacttcggttggggtaaggctattttcggtggtccaactactactggtgctagaatcactagaggtttggtttctttctgtgttccattcatgaacagaaacggtgagaagggtactgctttgtccttgtgtttgccaccaccagctatggaaagattcagagctaacgttcacgcttccttgcaggttaagcaagttgttgatgctgttgactcccacatgcagactattcaatccgcttccaagtaa

SEQ ID NO: 2 - protein, 468 aa, Cucumis melo
  REGION 1..468: note = CM-AAT1_AA
MHHHHHHETMQTIDFSFHVRKCQPELIAPANPTPYEFKQLSDVDDQQSLRLQLPFVNIYPHNPSLEGRDPVKVIKEAIGKALVFYYPLAGRLREGPGRKLFVECTGEGILFIEADADVSLEEFWDTLPYSLSSMQNNIIHNALNSDEVLNSPLLLIQVTRLKCGGFIFGLCFNHTMADGFGIVQFMKATAEIARGAFAPSILPVWQRALLTARDPPRITFRHYEYDQVVDMKSGLIPVNSKIDQLFFFSQLQISTLRQTLPAHLHDCPSFEVLTAYVWRLRTIALQFKPEEEVRFLCVMNLRSKIDIPLGYYGNAVVVPAVITTAAKLCGNPLGYAVDLIRKAKAKATMEYIKSTVDLMVIKGRPYFTVVGSFMMSDLTRIGVENVDFGWGKAIFGGPTTTGARITRGLVSFCVPFMNRNGEKGTALSLCLPPPAMERFRANVHASLQVKQVVDAVDSHMQTIQSASK

SEQ ID NO: 3 - DNA, 1383 bp, Malus pumila
  misc_feature 1..1383: note = MpAAT1
  source 1..1383: mol_type = genomic DNA
atgcatcaccaccaccatcactcattctccgtcttgcaggttaagagattgcagccagagttgatcactccagctaagtctactccacaagagactaagttcttgtccgacatcgacgaccaagagtccttgagagttcagatcccaatcatcatgtgctacaaggacaacccatccctgaacaagaacagaaacccagtcaaggctatcagagaggctttgtccagagccttggtttactactacccattggccggtagattgagagagggtccaaacagaaagttggtcgttgactgtaacggtgagggtatcttgttcgttgaagcttccgctgacgttaccttggaacaattgggtgacaagatcctgccaccatgtcctttgttggaagagttcctgtacaacttcccaggttccgacggtattatcgactgtccattgctgttgatccaggttacctgtttgacctgcggtggtttcatcttggccttgagattgaaccacactatgtgtgacgctgccggtttgttgttgttcttgactgctattgctgagatggctagaggtgctcacgctccatctattttgccagtttgggagagagagttgttgttcgctagagatccaccaagaatcacttgtgctcaccacgaatacgaggacgttattggtcactctgacggttcttacgcttcttccaaccagtccaacatggtccagagatccttttacttcggtgccaaagagatgagggtcctgagaaagcaaattccaccacacttgatctccacctgttccaccttcgacttgatcactgcttgtctgtggaagtgtagaaccttggccttgaacatcaacccaaaagaggccgttagagtctcctgtatcgttaacgctagaggtaagcacaacaacgtcagattgccattgggttactacggtaacgctttcgctttcccagctgctatttctaaggctgagccattgtgcaagaaccctttgggttacgctttggagttggtcaagaaagctaaggccaccatgaacgaagagtacttgagatccgttgccgacttgttggtcttgagaggtagaccacaatactcctccactggttcctacttgatcgtttccgacaacaccagagttggtttcggtgacgttaacttcggttggggtcaaccagtttttgccggtccagttaaggctttggacctgatctctttctacgtccaacacaagaacaacaccgaggacggtattttggtcccaatgtgtttgccatcctccgccatggaaagattccaacaagagttggagagaatcacccaagagccaaaagaggacatctgcaacaacttgagatccacctctcagtaa

SEQ ID NO: 4 - protein, 460 aa, Malus pumila
  REGION 1..460: note = MpAAT1_AA
MHHHHHHSFSVLQVKRLQPELITPAKSTPQETKFLSDIDDQESLRVQIPIIMCYKDNPSLNKNRNPVKAIREALSRALVYYYPLAGRLREGPNRKLVVDCNGEGILFVEASADVTLEQLGDKILPPCPLLEEFLYNFPGSDGIIDCPLLLIQVTCLTCGGFILALRLNHTMCDAAGLLLFLTAIAEMARGAHAPSILPVWERELLFARDPPRITCAHHEYEDVIGHSDGSYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRVGFGDVNFGWGQPVFAGPVKALDLISFYVQHKNNTEDGILVPMCLPSSAMERFQQELERITQEPKEDICNNLRSTSQ

SEQ ID NO: 5 - DNA, 1386 bp, Fragaria vesca
  misc_feature 1..1386: note = VAAT
  source 1..1386: mol_type = genomic DNA
atgcaccatcaccatcaccatgagaagattgaggtttcaatcatatccaaacacacaatcaaacccagcacgagcagttctcctttgcaaccatacaagctaaccttattggatcagttaactccaccgtcctacgtccctatggtgtttttctaccctattacgggtccagccgtgttcaatttgcaaacattggcagatctgagacacgcattgtctgagactttgacactgtactatccattatcgggtcgtgtgaaaaacaatctgtatattgatgattttgaagagggtgttccctacttggaagcgagagtgaactgcgacatgaatgactttttgaggcttccaaaaatcgaatgtttgaatgaatttgttccaatcaagcctttttctatggaggctatatccgatgaaagatatcctttacttggtgtgcaagtcaacattttcaattccgggatcgcaattggagttagcgtatcccataagttgatcgacggtcgtacatctgactgcttcctgaagtcatggtgtgctgtattccgaggatcacgtgacaaaatcatccatcccaatctttcgcaggcagctttgttgtttcctccgcgtgacgatctcccagagaaatatgctagacaaatggagggcttgtggtttgttggtaaaaaggttgctactagaagattcgtgttcggagcaaaggctatctctgttattcaagacgaggctaagtcagagtccgttccaaaaccatcaagagttcaagctgtcacatccttcttatggaaacaccttattgcaacttctagagctttaacttcgggtacaactagtactagactatccattgctacccaggtcgtcaacattagaagtaggagaaatatggaaacggtgtgggataatgccattggtaatcttatctggtttgctcctgcaatcttggaactgtctcatacaaccttggagatctccgatttgaaactgtgtgatctggttaacctactcaatggttccgtcaaacaatgtaatggcgattacttcgagacttttatgggtaaggaaggttatggatcaatgtgtgagtacttggacttccaacgtacaatgtccagcatggaaccagctccagaaatctaccttttcacttcatggaccaatttcttcaaccagctagactttggatggggtagaaccagctggattggtgtagctggaaagatagaaagtgctttttgtaacctgactacattggtacccactccttgcgatacaggaattgaggcatgggttaaccttgaagaggaaaagatggccatgttggaacaagaccctcagtttctggccttagcctctccaaaaactttgatatctaggtattaa

SEQ ID NO: 6 - protein, 461 aa, Fragaria vesca
  REGION 1..461: note = VAAT_AA
MHHHHHHEKIEVSIISKHTIKPSTSSSPLQPYKLTLLDQLTPPSYVPMVFFYPITGPAVFNLQTLADLRHALSETLTLYYPLSGRVKNNLYIDDFEEGVPYLEARVNCDMNDFLRLPKIECLNEFVPIKPFSMEAISDERYPLLGVQVNIFNSGIAIGVSVSHKLIDGRTSDCFLKSWCAVFRGSRDKIIHPNLSQAALLFPPRDDLPEKYARQMEGLWFVGKKVATRRFVFGAKAISVIQDEAKSESVPKPSRVQAVTSFLWKHLIATSRALTSGTTSTRLSIATQVVNIRSRRNMETVWDNAIGNLIWFAPAILELSHTTLEISDLKLCDLVNLLNGSVKQCNGDYFETFMGKEGYGSMCEYLDFQRTMSSMEPAPEIYLFTSWTNFFNQLDFGWGRTSWIGVAGKIESAFCNLTTLVPTPCDTGIEAWVNLEEEKMAMLEQDPQFLALASPKTLISRY

SEQ ID NO: 7 - DNA, 1404 bp, Cucumis melo
  misc_feature 1..1404: note = CM-AAT2
  source 1..1404: mol_type = genomic DNA
atgcatcaccatcaccaccacgagactatgcagactatcgacttctcattccaggttagaaagtgtcagccagagttgatcgctccagctaacccaactccatacgagttcaagcaattgtccgacgttgacgaccaacagtccttgagattccagttgccattggttaacatctaccaccacaacccatccttggagggtagagatccagttaaggttatcaaagaggctatcgctaaggctttggttttctactacccattggctggtagattgagagagggtcctggtagaaagttgttcgttgagtgtactggtgagggtatcttgttcattgaagctgacgctgacgtttccttggagcagttcagagatactttgccatactccttgtcctccatggaaaacaacatcatccacaactcattgaactccgacggtgttttgaactcccctttgttgttgatccaggttactagattgaagtgtggtggtttcatcttcggtatccacttcgaccacactatggctgacggttttggtatcgctcagttcatgaaggctattgctgagatcgctagaggtgctttcgctccatctattttgccagtttggcagagagctttgttgactgctagagatcctccaagaatcactgttagacactacgagtacgaccaggttgttgacactaagtccactttgatcccagctaacaacatgatcgacagattgttcttcttcactcagagacagatctccacattgagacagactttgccagctcacttgcacgactgttcttcattcgaggttttgactgcttacgtttggagattgagaactatcgctttccagttgaagccagaggaagaggttagattcttgtgtgttgttaacttgagatccaagatcgacatcccattgggtttctacggtaacgctatcgttttcccagctgttatcactactgttgctaagttgtgtggtaaccctttgggttacgctgttgacttgatcagaaaggctaaggctaaagctacaaaagagtacatcaagtccatggttgacttcatggttatcaagggtagaccaagattcactgagatcggtccattcatgatgtccgacattactagaatcggtttcgagaacgttgacttcggttggggtaaggctattttcggtggtccaattatcggtggttgtggtatcatcagaggtatgatctcttactccattgctttcatgaacagaaacggtgagaagggaatcgttgttccattgtgtttgccaccaccagctatggaaagattcagagctaacgttcacgcttccttgcaggttatccaggttttggacaaggttgacagagacatgcaaacaatcttgtccgctttgtaa

SEQ ID NO: 8 - protein, 467 aa, Cucumis melo
  REGION 1..467: note = CM-AAT2_AA
MHHHHHHETMQTIDFSFQVRKCQPELIAPANPTPYEFKQLSDVDDQQSLRFQLPLVNIYHHNPSLEGRDPVKVIKEAIAKALVFYYPLAGRLREGPGRKLFVECTGEGILFIEADADVSLEQFRDTLPYSLSSMENNIIHNSLNSDGVLNSPLLLIQVTRLKCGGFIFGIHFDHTMADGFGIAQFMKAIAEIARGAFAPSILPVWQRALLTARDPPRITVRHYEYDQVVDTKSTLIPANNMIDRLFFFTQRQISTLRQTLPAHLHDCSSFEVLTAYVWRLRTIAFQLKPEEEVRFLCVVNLRSKIDIPLGFYGNAIVFPAVITTVAKLCGNPLGYAVDLIRKAKAKATKEYIKSMVDFMVIKGRPRFTEIGPFMMSDITRIGFENVDFGWGKAIFGGPIIGGCGIIRGMISYSIAFMNRNGEKGIVVPLCLPPPAMERFRANVHASLQVIQVLDKVDRDMQTILSAL

SEQ ID NO: 9 - DNA, 1398 bp, Malus domestica
  misc_feature 1..1398: note = Md-AAT2
  source 1..1398: mol_type = genomic DNA
atgcatcaccatcaccaccacatgccattctccgttttgcaggttaagagattgcagttggagttgatcactcctgctaagccaacattgcaagaggctaagttcttgtccgacatcgacgaccaagagggtttgagattccaggttccagttatcatgtgttacaaggacaacccatccttgaacaagaactgtaacccagttaaggttatcagagaggctttgtccagagctttggtttactactacccattggctggtagattgaaagagggtccaaacagaaagttgatggttgactgtaacggtgagggtatcttgttcgttgaagcttccgctgacgttactttggagcaattgggtgacaagattttgccaccatgtcctttgttggaagagtttttgttcaacttcccaggttccgacggtatcatcggatgtcctttgttgttggttcaggttacttgtttgacttgtggtggtttcatcttggctttgagagttaaccacactatgtgtgacgctccaggtttgttgttgttcttgactgctatcgctgagatggctagaggtgctcatgctccatctattttgccagtttgggagagagagttgttgttttccagagatccaccaagaatcacttgtgctcaccacgaatacgaggacgttattgaccactctgacggtttgtacgcttcttccaaccagtccaacatggttcagagatccttctacttcggtgctaaagagatgagagttttgagaaagcagatcccaccacacttgatctccacttgttccactttcgacttgatcactgcttgtttgtggaagtgtagaactttggctttgaacatcaacccaaaagaggctgttagagtttcctgtatcgttaacgctagaggtaagcacaacaacgttagattgccattgggttactacggtaacgctttcgctttcccagctgctatttctaaggctgagccattgtgtaagaaccctttgggttacgctttggagttggttaagaaagctaaggctactatgaacgaagagtacttgagatccgttgctgacttgttggttttgagaggtagacctcagtactcctccactggatcctacttgatcgtttccgacaacactagagctggtttcggtgacgttaacttcggttggggtcaaccagtttttgctggtccagctaaagctttggacttgatttccttctacgttcaacacaagaacaatactgaggacggaattttggttccaatgtgtttgccatcctccgctatggaaagattccagcaagagttggagagaatcactcaagagcctaaagaggacatctgtaacaatttgagatccactagaattatgtccatgatgtaa

SEQ ID NO: 10 - protein, 465 aa, Malus domestica
  REGION 1..465: note = Md-AAT2_AA
MHHHHHHMPFSVLQVKRLQLELITPAKPTLQEAKFLSDIDDQEGLRFQVPVIMCYKDNPSLNKNCNPVKVIREALSRALVYYYPLAGRLKEGPNRKLMVDCNGEGILFVEASADVTLEQLGDKILPPCPLLEEFLFNFPGSDGIIGCPLLLVQVTCLTCGGFILALRVNHTMCDAPGLLLFLTAIAEMARGAHAPSILPVWERELLFSRDPPRITCAHHEYEDVIDHSDGLYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRAGFGDVNFGWGQPVFAGPAKALDLISFYVQHKNNTEDGILVPMCLPSSAMERFQQELERITQEPKEDICNNLRSTRIMSMM

SEQ ID NO: 11 - DNA, 1389 bp, Clarkia breweri
  misc_feature 1..1389: note = BEBT
  source 1..1389: mol_type = genomic DNA
atgcatcatcaccatcaccacgctcacgaccaatctttgtctttcgaggtctgcagaagaaagcccgagttgattagaccagctaagcaaactccacacgagttcaagaagttgtccgacgttgaagatcaagagggtctgagattccagatcccagtcatccaattctacaagcacaacaacgagtccatgcaagagagagatccagtccaggttatcagagagggtatcgctagagccttggtctactactacccattcgctggtagattgagagaggtcgacggtagaaagttggttgttgagtgtactggtgagggtgtcatgttcattgaagctgacgctgacgttaccttggagcaatttggtgatgcattgcagccaccattcccatgtttcgaccagttgttgttcgacgttccaggttccggtggtattttggactctccattgctgttgatccaggtcaccagattgaagtgcggttccttcatcttcgccttgagattgaaccacactatggctgatgctgccggtatcgtcttgttcatgaaggctgttggtgagatggctagaggtgctgctactccatctactttgccagtttgggacagacacatcttgaacgctagagttccaccacaggttaccttcaaccacagagagtacgaagaggtcaagggaactatcttcactccattcgatgacttggcccacagatcctttttcttcggttccactgaaatctccgccatgagaaagcaaatcccaccacacttgagatcctgttccactaccatcgaggttttgactgcttgtttgtggcgttgtagaaccttggccattaagccaaacccagacgaagaggtgagaatgatctgtatcgttaacgccagatccaagttcaacccaccattgccagatggttactacggtaacgctttcgctattccagctgctgttactactgccggtaagttgtgtaacaacccattgggtttcgccttggagttgatcagaaaggccaagagagaggtcaccgaagagtacatgcattccgttgctgacttgatggttgctactggtagaccacacttcaccgttgtcaacacctacttggtttccgacgttactagagctggtttcggtgaagttgatttcggttggggtgaagctgtttacggtggtccagctaaaggtggtgttggtgttattccaggtgtcacctccttctacatcccactgagaaacagacaaggtgagaagggtatcgttctgccaatctgtttgccatccgctgccatggaaattttcgctgaggctttgaacaacaccctgaacggtaaagagatcgagatcgctaagcacttcactcagtcctccctgtaa

SEQ ID NO: 12 - protein, 462 aa, Clarkia breweri
  REGION 1..462: note = BEBT_AA
MHHHHHHAHDQSLSFEVCRRKPELIRPAKQTPHEFKKLSDVEDQEGLRFQIPVIQFYKHNNESMQERDPVQVIREGIARALVYYYPFAGRLREVDGRKLVVECTGEGVMFIEADADVTLEQFGDALQPPFPCFDQLLFDVPGSGGILDSPLLLIQVTRLKCGSFIFALRLNHTMADAAGIVLFMKAVGEMARGAATPSTLPVWDRHILNARVPPQVTFNHREYEEVKGTIFTPFDDLAHRSFFFGSTEISAMRKQIPPHLRSCSTTIEVLTACLWRCRTLAIKPNPDEEVRMICIVNARSKFNPPLPDGYYGNAFAIPAAVTTAGKLCNNPLGFALELIRKAKREVTEEYMHSVADLMVATGRPHFTVVNTYLVSDVTRAGFGEVDFGWGEAVYGGPAKGGVGVIPGVTSFYIPLRNRQGEKGIVLPICLPSAAMEIFAEALNNTLNGKEIEIAKHFTQSSL

SEQ ID NO: 13 - DNA, 1320 bp, Clarkia breweri
  misc_feature 1..1320: note = CbBEAT
  source 1..1320: mol_type = genomic DNA
atgcatcatcaccaccaccacaacgttactatgcactccaagaagttgctgaagccatccatcccaactccaaaccacttgcagaagttgaacttgtccttgctggaccagatccagatcccattctacgtcggtttgatcttccactacgagactttgtctgacaactccgacatcaccttgtccaagttggaatcttccttgtccgagactctgaccttgtactaccatgttgccggtagatacaacggtactgactgtgttatcgagtgcaacgaccagggtattggttacgttgaaactgccttcgacgttgagttgcaccagttcttgttgggtgaagagtccaacaacttggacttgttggttggtttgtccggtttcttgtccgaaactgagactccaccattggctgctatccagctgaacatgtttaagtgcggtggtttggttatcggtgcccagttcaaccacattatcggtgacatgttcaccatgtccaccttcatgaactcttgggctaaggcctgtagagtcggtatcaaagaagttgctcacccaactttcggtttggccccattgatgccatctgccaaggttttgaacattccaccaccaccatctttcgagggtgtcaagttcgtttccaagaggttcgtgttcaacgagaacgccatcaccagattgagaaaagaggctactgaagaggacggtgatggtgatgacgaccaaaagaagaagaggccatccagagttgacttggttactgccttcttgtccaagtccttgatcgagatggactgcgctaagaaagagcagactaagtccagaccatccctgatggttcacatgatgaacctgagaaagagaactaagctggccttggagaacgacgtttccggtaacttcttcatcgttgttaacgccgagtccaagatcactgttgctccaaagatcactgacttgaccgaatctttgggttccgcttgtggtgagattatctccgaggttgctaaggttgacgacgctgaagttgtttcctccatggttttgaactccgtccgtgagttctactacgaatggggtaagggtgagaagaacgtgttcttgtacacctcctggtgtagattcccactgtacgaagttgatttcggttggggtatcccatccttggttgacactactgctgttccattcggtctgatcgttttgatggatgaagctccagctggtgacggtattgctgttagagcttgtttgtctgagcacgacatgatccaattccaacagcaccaccagttgctgtcctacgtttcttaa

SEQ ID NO: 14 - protein, 439 aa, Clarkia breweri
  REGION 1..439: note = CbBEAT_AA
MHHHHHHNVTMHSKKLLKPSIPTPNHLQKLNLSLLDQIQIPFYVGLIFHYETLSDNSDITLSKLESSLSETLTLYYHVAGRYNGTDCVIECNDQGIGYVETAFDVELHQFLLGEESNNLDLLVGLSGFLSETETPPLAAIQLNMFKCGGLVIGAQFNHIIGDMFTMSTFMNSWAKACRVGIKEVAHPTFGLAPLMPSAKVLNIPPPPSFEGVKFVSKRFVFNENAITRLRKEATEEDGDGDDDQKKKRPSRVDLVTAFLSKSLIEMDCAKKEQTKSRPSLMVHMMNLRKRTKLALENDVSGNFFIVVNAESKITVAPKITDLTESLGSACGEIISEVAKVDDAEVVSSMVLNSVREFYYEWGKGEKNVFLYTSWCRFPLYEVDFGWGIPSLVDTTAVPFGLIVLMDEAPAGDGIAVRACLSEHDMIQFQQHHQLLSYVS

SEQ ID NO: 15 - DNA, 1377 bp, Fragaria X ananassa
  misc_feature 1..1377: note = SAAT
  source 1..1377: mol_type = genomic DNA
atgcatcatcaccaccaccacgagaagatcgaggtttccattaactccaagcacactatcaagccttccacttcctccactccattgcagccatacaagttgactttgttggaccagttgactccaccagcttacgttccaatcgttttcttctacccaatcactgaccacgacttcaacttgccacaaactttggctgacttgagacaggctttgtccgagactttgactttgtactacccattgtccggtagagttaagaacaacttgtacatcgacgacttcgaagagggtgttccatacttggaggctagagttaactgtgatatgactgacttcttgagattgagaaagatcgagtgtttgaacgagttcgttccaatcaagccattctccatggaagctatctccgacgagagataccctttgttgggtgttcaggttaacgttttcgactccggtatcgctatcggtgtttctgtttcccacaagttgatcgacggtggtactgctgactgtttcttgaagtcttggggtgctgttttcagaggttgtagagagaacatcatccacccatctttgtccgaggctgctttgttgtttccaccaagagatgacttgccagagaagtacgttgaccagatggaagctttgtggttcgctggtaagaaggttgctactagaagattcgttttcggtgttaaggctatctcctccattcaggacgaagctaagtctgagtctgttccaaagccatccagagttcacgctgttactggtttcttgtggaagcacttgatcgctgcttccagagctttgacttctggtactacttccactagattgtccattgctgctcaggctgttaacttgagaactagaatgaacatggaaactgttttggacaacgctactggtaacttgttctggtgggctcaggctattttggagttgtctcacactactccagagatctccgacttgaagttgtgtgacttggttaacttgttgaacggttccgttaagcagtgtaacggtgactacttcgagactttcaagggtaaagagggttacggtagaatgtgtgagtacttggacttccagagaactatgtcctccatggaaccagctccagatatctacttgttctcctcctggactaacttcttcaacccattggatttcggttggggtagaacttcctggattggtgttgctggtaagattgagtccgcttcctgtaagttcatcattttggttccaactcagtgtggttccggtatcgaagcttgggttaacttggaagaagagaagatggctatgttggagcaagacccacacttcttggctttggcttctccaaagactttgatctaa

SEQ ID NO: 16 - protein, 458 aa, Fragaria X ananassa
  REGION 1..458: note = SAAT_AA
MHHHHHHEKIEVSINSKHTIKPSTSSTPLQPYKLTLLDQLTPPAYVPIVFFYPITDHDFNLPQTLADLRQALSETLTLYYPLSGRVKNNLYIDDFEEGVPYLEARVNCDMTDFLRLRKIECLNEFVPIKPFSMEAISDERYPLLGVQVNVFDSGIAIGVSVSHKLIDGGTADCFLKSWGAVFRGCRENIIHPSLSEAALLFPPRDDLPEKYVDQMEALWFAGKKVATRRFVFGVKAISSIQDEAKSESVPKPSRVHAVTGFLWKHLIAASRALTSGTTSTRLSIAAQAVNLRTRMNMETVLDNATGNLFWWAQAILELSHTTPEISDLKLCDLVNLLNGSVKQCNGDYFETFKGKEGYGRMCEYLDFQRTMSSMEPAPDIYLFSSWTNFFNPLDFGWGRTSWIGVAGKIESASCKFIILVPTQCGSGIEAWVNLEEEKMAMLEQDPHFLALASPKTLI

SEQ ID NO: 17 - DNA, 1194 bp, Fragaria X ananassa
  misc_feature 1..1194: note = Fa-AAT2
  source 1..1194: mol_type = genomic DNA
atgcatcatcaccaccaccactcctacaagaacaaccactccattttgtccaagccaaacgacccagttgaggttatcagagatgctttgtccaaggctttgcagttctactacccattggctggtagattgagagagggtccaaacaagaaattgatggttgactgtactggtgagggtatcttgttcgttgaagctaacgctgaggttactttggacgaattgggtgacgctatcttgccaccatgtccattcttggacggtttcttgttcaacgttccaggttccgacggtattttgggttccccattgtgtttgatccaggttactagattgtcctgtggtggtttcatcttcgctttgagattgaaccacactatctgtgacgctttgggtttggttcagttcttgaacgctgttggtgagatcgctcagggtaaatacgctccatccattactccagtttgggagagagagttgttgtccgctagagatccaccaagaatctcttgtactcacgaagagttcgacgactccattgaccactcttacccaaactacggtgctactgttcagcagtgttactgtttcggtccaaaagagatcaagtccttgagagagcatttgccaccacacttgtctacttgttcctccactttcgagttgatcactgcttgtgtttggaagtgtagaactatctccttggacatggacccagagcagatcgttagattgtcttgtgttgttactgctttgggtaagcacaacaacgtttgtttgccattgggttactacggtaacactttcacttacccagctgttgtttccactgctgagagattgtgtaactcccctttgggttacgctgttgagttggttaagaaatccaaggctaagatgtccgaagagtacttgagatccgctatcgacttcgttgaggttagaggtagaccaccattcgctttggaaggtatgtccgacttcttggtttccgacaacactagaactggtttgggtgagatcgacttcggtttcggtaagccagtttacgctggtgttgctaagtccactgacttgatctcattctacgttagatccactaacaaagaagagagagagattttggttccagtttgtttgcctatcttgtccatggaaatcttccagcaagagttgaagaagatgatcggttag

SEQ ID NO: 18 - protein, 397 aa, Fragaria X ananassa
  REGION 1..397: note = Fa-AAT2_AA
MHHHHHHSYKNNHSILSKPNDPVEVIRDALSKALQFYYPLAGRLREGPNKKLMVDCTGEGILFVEANAEVTLDELGDAILPPCPFLDGFLFNVPGSDGILGSPLCLIQVTRLSCGGFIFALRLNHTICDALGLVQFLNAVGEIAQGKYAPSITPVWERELLSARDPPRISCTHEEFDDSIDHSYPNYGATVQQCYCFGPKEIKSLREHLPPHLSTCSSTFELITACVWKCRTISLDMDPEQIVRLSCVVTALGKHNNVCLPLGYYGNTFTYPAVVSTAERLCNSPLGYAVELVKKSKAKMSEEYLRSAIDFVEVRGRPPFALEGMSDFLVSDNTRTGLGEIDFGFGKPVYAGVAKSTDLISFYVRSTNKEEREILVPVCLPILSMEIFQQELKKMIG

SEQ ID NO: 19 - DNA, 1317 bp, Fragaria X ananassa
  misc_feature 1..1317: note = AeAT9
  source 1..1317: mol_type = genomic DNA
atgcaccatcaccatcaccatgcctcctccgtccgtctagtcaaaaagcctgttttagtggcaccagttgacccaacaccatcaaccgtcttgagcctttcctctctagactcgcaactgttcctccgattccccattgagtatctgcttgtctatgcttctccacatggagtggatagggcagtcactgctgcaagggtaaaagcagcactggcaagatcattagtgccatactatcctttggctggacgtgtgaaaactagaccggattctactggattagacgttgtctgtcaagctcaaggtgctggtttgctggaggcagtttctgactacacggctagtgactttcaaagagcccccagatctgttacagaatggaggaagctgctgttggtcgaagtctttaaggttgtacctccactggtggttcaattaacttggttatcagatggttgtgtagctttgggtgttggcttcagtcactgtgtaatcgatggaattggttcaagtgagtttttgaacctttttgctgagctagccacaggtagagctagattgagcgaatttcagccaaaacccgtttgggatagacatttactcaatagcgctggtagaacaaatcttggtactcaccccgagttcggacgtgtgcctgatttgtcagggttcgttacccgtttcactcaggaaagactttcccctacctcgatcacatttgataagacatggttgaaagagttgaaaaatattgcaatgtccacttcacaacctggcgagttcccatacacatcctttgaggtattgagcggacatatctggcgtagttgggcccgttcgttgaatttgccagctaaacaggtattgaaactactcttctccataaacatcagaaacagagttaagccttctttgcctgcgggatactatggtaatgcatttgttctgggttgtgctcaaacatccgttaaggatcttactgagaaaggattgggttactgtgctgacttggtcagaggtgctaaggaaagagtgggtgatgaatatgccagggaagtcgttgagtcagtgagttggccacgtagagcatccccggactccgtgggtgtgttgatcattagccaatggtctagattaggactggaccgtgttgactttggtttgggccgtcctgtacaggtgggtccaatttgttgcgatagatattgccttttcttgcctgttagggaatccacggaatctgtgaaggttatggttgctgttccaacttctgctgttgacagatacgaatacttcatcagatcaccatactcctag

SEQ ID NO: 20 - protein, 438 aa, Fragaria X ananassa
  REGION 1..438: note = AeAT9_AA
MHHHHHHASSVRLVKKPVLVAPVDPTPSTVLSLSSLDSQLFLRFPIEYLLVYASPHGVDRAVTAARVKAALARSLVPYYPLAGRVKTRPDSTGLDVVCQAQGAGLLEAVSDYTASDFQRAPRSVTEWRKLLLVEVFKVVPPLVVQLTWLSDGCVALGVGFSHCVIDGIGSSEFLNLFAELATGRARLSEFQPKPVWDRHLLNSAGRTNLGTHPEFGRVPDLSGFVTRFTQERLSPTSITFDKTWLKELKNIAMSTSQPGEFPYTSFEVLSGHIWRSWARSLNLPAKQVLKLLFSINIRNRVKPSLPAGYYGNAFVLGCAQTSVKDLTEKGLGYCADLVRGAKERVGDEYAREVVESVSWPRRASPDSVGVLIISQWSRLGLDRVDFGLGRPVQVGPICCDRYCLFLPVRESTESVKVMVAVPTSAVDRYEYFIRSPYS

SEQ ID NO: 21 - DNA, 1392 bp, Fragaria X ananassa
  misc_feature 1..1392: note = Rh-AAT1
  source 1..1392: mol_type = genomic DNA
atgcatcaccatcaccaccacgagaagattgaggtttccatcatctccagagacactatcaagccttccgctgcttcttcttccttgcacccatacaagttgtccatcatcgaccagttcactccaactacttacttcccagttatcttcttctacccaatcactgacagagttttcaacttgccacagactttgactgacttgaagaacactgtttcccaggctttgactttgtaccacccattgtccggtagaatcaagaacaacttgtacatcgacgacttcgaggctggtatcccatacttggaagctagagttaacttccacatgatcgatttcttgagattgccaaagatcgagtggttgaacgagttcgttccaatggctccatacagaaaagagactatctccgagttcttgcctttgttgggtatccaggttaacatcttcgactccggtatcgctatcggtgtttcattctcccacaagatcaacgacggtcagactgcttcctgtttcttgaagtcctgggttgctatcttcagaggttacagaaacaagatcatccacccaaacttgtcccaggctgctttgttgttgccatccagagatgatttgccagagaagtacgttgctatgatggaaagaatgtggttcggtgagaagaaggttgttactagaagattcgttttcgacgctaaggctatctccgctttgcaagatgagggaaagtctgagtacgttccaaagccttccagagttcaagctttgactggtttcttgtggaagcaccagttggctgcttctagagctttgtcctctggtacttccactagattctccgttgcttcccagactgttaacttgagatccaagatgaacatgaagactactttggacaacgctatcggaaatatcttcttgtgggcttccgctagattggacttgaacgatactgctccaggttcctccgacttgaagttgtgtgacttggttaacttgttgaacgaatccatcaaagagttcaactccgattacttggagatcttgaagggtaaagagggttacggtggtatgtgtgatttgttggacttcatggaagagggttccttcgttgaaccagctccagagttttactcattctcctcatggacaagattcttcgaccaggttgatttcggttggggtagaccatcttgggttggtttctctggtagagttgagactagaaacttcactatcttcgttgagactcagtgtgacgacggtattgacgcttgggttactgttgacgagaagcagatggctatgttggagcaagacccacagtttttggctttcgcttctccaaacccaagaatctctatcgcttcctccgttggtatggactag

SEQ ID NO: 22 - protein, 463 aa, Fragaria X ananassa
  REGION 1..463: note = Rh-AAT1_AA
MHHHHHHEKIEVSIISRDTIKPSAASSSLHPYKLSIIDQFTPTTYFPVIFFYPITDRVFNLPQTLTDLKNTVSQALTLYHPLSGRIKNNLYIDDFEAGIPYLEARVNFHMIDFLRLPKIEWLNEFVPMAPYRKETISEFLPLLGIQVNIFDSGIAIGVSFSHKINDGQTASCFLKSWVAIFRGYRNKIIHPNLSQAALLLPSRDDLPEKYVAMMERMWFGEKKVVTRRFVFDAKAISALQDEGKSEYVPKPSRVQALTGFLWKHQLAASRALSSGTSTRFSVASQTVNLRSKMNMKTTLDNAIGNIFLWASARLDLNDTAPGSSDLKLCDLVNLLNESIKEFNSDYLEILKGKEGYGGMCDLLDFMEEGSFVEPAPEFYSFSSWTRFFDQVDFGWGRPSWVGFSGRVETRNFTIFVETQCDDGIDAWVTVDEKQMAMLEQDPQFLAFASPNPRISIASSVGMD

SEQ ID NO: 23 - DNA, 1458 bp, Cucumis melo
  misc_feature 1..1458: note = CM-AAT4
  source 1..1458: mol_type = genomic DNA
atgcatcaccatcaccaccacgaggttaaggttttgtccaaagagactatcatcccatcctccccaactccaccacacttgcaaccattgaacttgtccttgttggaccagttgtccccaatgttgtacatccctttgttgttgttctacccaatgaagaagtcctaccagcaccaggatcacaacaaggctatcgctactttgaaaacttccttgtccaagactttgtccagattctacttgttggctggtagaatcatcggtaagtccatccactgtaacgacaagggtgctgttttcatggaagctactatcaactccaacatgttcgacatcttgaaagagccaaacaacgaggttttgactaagttgttgccatgttctttgttgtgtaacactaagccaatcgaagagtacccacagatcgttgttcaggctaacatcttcgaatgtggtggtatcgctatctccttgtgtttgttgcacaagttgatcgacgctgctactttctgttgtttcttgagatcctgggctactacaaacagagagttgttgtctttggaccactcttccccaaacaacaatatggtttgtgttgactacaagtccttctcctccttgttcccacaaacaaacttgttgcctttccaccagtccttgatcaacaacgataaggctgttgttccaccatcctccatctttaacagaaagagaagattccagagattcgttttcagatccgaggctatcttggacttgaaggctaaggctaagtcctgtgacatcccaaacccaacttgtgttgagactttgacttgtttcatctggaagtacttgatgaaggttgctgacgacggtgactctcaaagaccatctactttgtcccacgttgttaacatcagaaagatgttggagccatccttgggtgaggtttctttgggtaacatcatgtggggtactgttgctcaccacttctccactactagaaacgaagagttcgagggtttggagttgtccaagttggtttccttgttgagacagtccttcaagaagattaacaaggactacatcaaagaattgatcatgggtggtgacaaagaaagaagaaacggtgttatgaagttggttggtgagatcaacaagtggccaatctccaactactacttcttcacttcctggaagaatttgaagttgaacgaggttgacttcggttggggtaagccattgtggtctgctattgctggtgacccaaacgagatgatgggaaacattatcgttttggttgacaacgttttggacgacggttctactgaggcttggattttgttggacgagaaagagatgcagttgttggagcagatcccacagtttttggagttcgctttgttgaacccatccatcaacttgccacacaaccagaaaactgctgacgagattttctccaacaaattgatctaa

SEQ ID NO: 24 - protein, 485 aa, Cucumis melo
  REGION 1..485: note = CM-AAT4_AA
MHHHHHHEVKVLSKETIIPSSPTPPHLQPLNLSLLDQLSPMLYIPLLLFYPMKKSYQHQDHNKAIATLKTSLSKTLSRFYLLAGRIIGKSIHCNDKGAVFMEATINSNMFDILKEPNNEVLTKLLPCSLLCNTKPIEEYPQIVVQANIFECGGIAISLCLLHKLIDAATFCCFLRSWATTNRELLSLDHSSPNNNMVCVDYKSFSSLFPQTNLLPFHQSLINNDKAVVPPSSIFNRKRRFQRFVFRSEAILDLKAKAKSCDIPNPTCVETLTCFIWKYLMKVADDGDSQRPSTLSHVVNIRKMLEPSLGEVSLGNIMWGTVAHHFSTTRNEEFEGLELSKLVSLLRQSFKKINKDYIKELIMGGDKERRNGVMKLVGEINKWPISNYYFFTSWKNLKLNEVDFGWGKPLWSAIAGDPNEMMGNIIVLVDNVLDDGSTEAWILLDEKEMQLLEQIPQFLEFALLNPSINLPHNQKTADEIFSNKLI

SEQ ID NO: 25 - DNA, 669 bp, Staphylococcus sciuri
  misc_feature 1..669: note = ACT
  source 1..669: mol_type = genomic DNA
atgcatcatcaccatcaccacaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataa

SEQ ID NO: 26 - protein, 222 aa, Staphylococcus sciuri
  REGION 1..222: note = ACT_AA
MHHHHHHNFNKIDLDNWKRKEIFNHYLNQQTTFSITTEIDISVLYRNIKQEGYKFYPAFIFLVTRVINSNTAFRTGYNSDGELGYWDKLEPLYTIFDGVSKTFSGIWTPVKNDFKEFYDLYLSDVEKYNGSGKLFPKTPIPENAFSLSIIPWTSFTGFNLNINNNSNYLLPIITAGKFINKGNSIYLPLSLQVHHSVCDGYHAGLFMNSIQELSDRPNDWLL

SEQ ID NO: 27 - DNA, 1278 bp, Fragaria X ananassa
  misc_feature 1..1278: note = BanAAT
  source 1..1278: mol_type = genomic DNA
atgcatcaccaccaccaccattccttcgctgttactagaacttccagatccttggttaccccatgtggtgttactccaactggttctttgggtttgtccgccattgatagagtcccaggtttgagacacatggtcagatccttgcacgttttcagacaaggtagagagccagccagaatcatcagagaagctttgtccaaggccctggtcaagtactacccatttgctggtagattcgttgacgatcctgaaggtggtggtgaggttagagttgcttgtactggtgaaggtgcctggttcgttgaagctaaggctgactgttctttggaggacgtcaagtacttggacctgccattgatgattccagaggacgctttgttgccaaagccatgtccaggtttgaacccattggacttgcctttgatgttgcaggttaccgagtttgtcggtggtggtttcgttgttggtttgatctccgttcacactatcgctgacggtttgggtgttgtccagttcattaacgctgttgctgagatcgctagaggtttgccaaagcctactgttgaaccagcttggtccagagaggttattccaaacccaccaaagttgccaccaggtggtccaccagtttttccatcctttaagttgttgcacgccaccgttgatttgtccccagatcacattgaccacgtcaagtctagacacttggagttgactggtcagagatgttccactttcgacgttgctatcgctaacttgtggcagtccagaactagagccattaacttggatccaggtgttgacgtccacgtctgtttcttcgctaacaccagacacttgttgagacaggtcgttttgttgccaccagaggatggttactacggtaactgtttctacccagttactgctactgctccctccggtagaattgcttctgctgagttgattgacgtcgtgtccatcatcagagatgccaagtctagattgccaggtgagtttgctaaatgggctgctggtgatttcaaggacgacccatacgagttgtcctttacctacaactccctgttcgtttccgactggactagattgggtttcttggacgttgattacggttggggtaagccattgcacgttatcccattcgcttacttggacatcatggccgttggtattattggtgctccaccagctccacaaaagggtactagagttatggctcagtgcgtcgagaaagaacacatgcaagctttcttggaagagatgaagggtttcgcttaa

SEQ ID NO: 28 - protein, 425 aa, Fragaria X ananassa
  REGION 1..425: note = BanAAT_AA
MHHHHHHSFAVTRTSRSLVTPCGVTPTGSLGLSAIDRVPGLRHMVRSLHVFRQGREPARIIREALSKALVKYYPFAGRFVDDPEGGGEVRVACTGEGAWFVEAKADCSLEDVKYLDLPLMIPEDALLPKPCPGLNPLDLPLMLQVTEFVGGGFVVGLISVHTIADGLGVVQFINAVAEIARGLPKPTVEPAWSREVIPNPPKLPPGGPPVFPSFKLLHATVDLSPDHIDHVKSRHLELTGQRCSTFDVAIANLWQSRTRAINLDPGVDVHVCFFANTRHLLRQVVLLPPEDGYYGNCFYPVTATAPSGRIASAELIDVVSIIRDAKSRLPGEFAKWAAGDFKDDPYELSFTYNSLFVSDWTRLGFLDVDYGWGKPLHVIPFAYLDIMAVGIIGAPPAPQKGTRVMAQCVEKEHMQAFLEEMKGFA

SEQ ID NO: 29 - DNA, 1299 bp, Zea mays
  misc_feature 1..1299: note = Glossy2
  source 1..1299: mol_type = genomic DNA
atgcatcatcaccaccaccacgttttcgaacaacacgaagaagaggctgttgctccaggtgctgttcatggtcatagattgtctaccgttgttccatcctccgttactggtgaagttgactacgctttggctgatgctgacttggctttcaagttgcactacctgagaggtgtctactactacagatctggtgacggtttggccaccaaggttttgaaggatccaatgttgccatggctggatgaccactttccagttgctggtagagttagaagggctgaaactgaaggtgatggtgctccaagacgtccttacatcaagtgtaacgactgcggtgttagaatcgttgaggccagatgtgatagagacatggctgagtggattagagatgctgctccaggtagaatcagacagttgtgttacgacaaggtcttgggtccagagttgttcttctccccattgctgtacgttcagatcaccaacttcaagtgtggtggtttggctttgggtttctcttgggctcacttgattggtgacattccatccgctgctacctgctttaacaagtgggctcaaatcctgtccggtaagaagccagaagctactgttttgactccaccaaaccagccattgcaaggtcaatctccagctgctccaagatccgttaagcaggttggtccaattgaggacttgtggttggttccagctggtagagatatggcctgttactctttccacgtttccgacgccgttttgaagaagttgcaccaacaacagaacggtagacaggatgctgctgctggtactttcgaattggtttccgctttggtttggcaggctgttgctaagattagaggtgacgttgacaccgttaccgttgttagagctgatgctgctggtagatctggtaagtctttggccaacgagatgaaggttggttacgttgaatctgctggatcctccccagctaagactgatttggctgaattggctgctttgctggccaagaacttggttgacgaaactgctgctgttgctgctttccaaggtgacgttttggtttacggtggtgccaacttgaccttggttgacatggaacaggttgacctgtacggtttggagatcaagggtcaaagaccagttcacgtcgaatacggtatggatggtgttggtgatgagggtgctgttttggttcaaccagatgctgatggtagaggtagattggttactgccgttttgccaggtgacgagattgactctttgagagctgctttgggttccgccttgcaggttgcttaa

SEQ ID NO: 30 - protein, 432 aa, Zea mays
  REGION 1..432: note = Glossy2_AA
MHHHHHHVFEQHEEEAVAPGAVHGHRLSTVVPSSVTGEVDYALADADLAFKLHYLRGVYYYRSGDGLATKVLKDPMLPWLDDHFPVAGRVRRAETEGDGAPRRPYIKCNDCGVRIVEARCDRDMAEWIRDAAPGRIRQLCYDKVLGPELFFSPLLYVQITNFKCGGLALGFSWAHLIGDIPSAATCFNKWAQILSGKKPEATVLTPPNQPLQGQSPAAPRSVKQVGPIEDLWLVPAGRDMACYSFHVSDAVLKKLHQQQNGRQDAAAGTFELVSALVWQAVAKIRGDVDTVTVVRADAAGRSGKSLANEMKVGYVESAGSSPAKTDLAELAALLAKNLVDETAAVAAFQGDVLVYGGANLTLVDMEQVDLYGLEIKGQRPVHVEYGMDGVGDEGAVLVQPDADGRGRLVTAVLPGDEIDSLRAALGSALQVA

SEQ ID NO: 31 - DNA, 1398 bp, Cucumis melo
  misc_feature 1..1398: note = CM-AAT3
  source 1..1398: mol_type = genomic DNA
atgcatcatcatcaccaccacgcatcctccctggttttccaagttcaaagatcccagccacagttgattccaccatctgatccaactccacacgagttcaagcagttgtctgacattgacgaccaagagggtctgagattccagatcccagttatccagttctacagacacgacccaagaatggctggtactgatccagccagagttatcaaagaggctatcgctaaggccctggttttctactacccattcgctggtagattgagagagggtccaggtagaaagttgttcgttgagtgtactggtgagggtgtcatgttcattgaagctgacgctgacgtttccttggagcaatttggtgatgcattgcagccaccattcccatgtttggaggaacctttgttcgacgttccaaactcctctggtgttttggactgtccattgctgttgatccaggtcaccagattgaagtgcggtggtttcatcttcgccttgagattgaaccacactatgtctgacgcttccggtttggtccaattcatgatggctgttggtgagatggctagaggtgctactgctccatctgttagaccagtttggcagagagctttgctgaacgctagagatccaccaaaggttacctgtcaccacagagaatacgacgaggttgttgacaccaagggtactatcattccattggacgacatggcccacagatccttttttttcggtccatccgaaatctccgccatcagaaaggctttgccatcccacttgagacagtgttcctcattcgaggttttgaccgcttgtctgtggcgtttcagaactatttccttgcaaccagacccagaggaagaggttagagttttgtgtatcgtcaactccagatccaagttcaacccaccattgccaactggttactacggtaacgctttcgctttcccagttgctttgactactgccggtaagttgtgtcagaacccattgggttacgccttggagttggttagaaaggctaaggctgatgtcaccgaggactacatgaagtctgttgccgacttgatggtcatcaagggtagaccacacttcaccgttgtcagaacctacttggtttccgacgttactagagctggtttcgaggatgttgatttcggttggggtaaggctatgtacggtggtccagctaaaggtggtgttggtgctattccaggtgttgcctctttctacatcccattcaagaacaagaagggcgagagaggtatcttggtcccattgtgtttaccagctccagccatggaaagattcgtcaaagaattggacgccttgctgaaggctggtaagactattgatggtgtcgacaacaagaagcccctgttcattgcttccgccttgtaa

SEQ ID NO: 32 - protein, 465 aa, Cucumis melo
  REGION 1..465: note = CM-AAT3_AA
MHHHHHHASSLVFQVQRSQPQLIPPSDPTPHEFKQLSDIDDQEGLRFQIPVIQFYRHDPRMAGTDPARVIKEAIAKALVFYYPFAGRLREGPGRKLFVECTGEGVMFIEADADVSLEQFGDALQPPFPCLEEPLFDVPNSSGVLDCPLLLIQVTRLKCGGFIFALRLNHTMSDASGLVQFMMAVGEMARGATAPSVRPVWQRALLNARDPPKVTCHHREYDEVVDTKGTIIPLDDMAHRSFFFGPSEISAIRKALPSHLRQCSSFEVLTACLWRFRTISLQPDPEEEVRVLCIVNSRSKFNPPLPTGYYGNAFAFPVALTTAGKLCQNPLGYALELVRKAKADVTEDYMKSVADLMVIKGRPHFTVVRTYLVSDVTRAGFEDVDFGWGKAMYGGPAKGGVGAIPGVASFYIPFKNKKGERGILVPLCLPAPAMERFVKELDALLKAGKTIDGVDNKKPLFIASAL

SEQ ID NO: 33 - DNA, 1464 bp, Fragaria X ananassa
  misc_feature 1..1464: note = BAHDFox
  source 1..1464: mol_type = genomic DNA
atgcatcatcaccatcaccacccatccactttgaacttccaatccgagactccaactgttcaaggtgaacaagacccatccttggttaccttggaacactacggattgcaaccaccatccgctcaacaaaagttgaccccattggacatgaacatgccaagactgtacggtatcagactgatcttgtgcttcccaactaacccaggtatggacaagagacagatctacgagaacctgaagaagggtttggctcacaccgttacttccgttccatggatttctggtcacatcggtccagaagaaggtcaggatccaaagaccagaaaggtccagattttggactccccatacggtttcagattcccatacaaggacttgactgacgccttgccaccatacgctgaattgcaagaaagaaacttcccactggctgagttcactactgctcaggttggtccaattgacgttatgccacaaggtccaaaccagccagtttttgctgctcaggctaacttcgttaagggtggtttgttgttgaccgttggtgttcaccactctgcttgtgatgctttggctttggacgctatcttgtctacttggtcccacaacactgctgttgcttcaggtggttctggttccttctctactttggacggtccatctaacgacagatccccattgatggaaggtgacttgggtaacgctgatgttgctgctttccctgagtacgttttgatgccaactccacactctactgagggtgacttgtcctctatgtccggttttcaaatgccaccattggcctctagactgttccacttctcaccagagtccctgagaaagttgaaggctgaagctggtgctttctcttctcacgatgctttgtgtgctttcatctggcagagaatgaccttggctagaatgcactccggtatcttcaacgatccaccaggtgacttgacctccagattctgtttcgccgtcaacatcagaaacagaatgtccccaccattgccaccatcctacatgggtaatgcttccatgggttgtgtcaccgagaagatttctgttgcctccatgatctccaacaacggtttgaagcaagcctccgtcactatcagaagatccctgaacgatttcaactccccaagaagggctacttccaccatcggtttgttgagatctagaccagacccaaccgacttcaagttgtccttcaacggtttcttgggtccagacgttgttgaatcttcttgggctgacttgggtgtttacggtcatcaatggggtgacgctattggtactttggacgctgttagaatcccaggtgaaggttctgacggtactatgatgatcctgccaagattgaaggacggtggtttggacgttgttgttggtttgtctactgctgccatggaaagactgttggaggacgaaaagttcgtttccgttgctcactcttaa

SEQ ID NO: 34 - protein, 487 aa, Fragaria X ananassa
  REGION 1..487: note = BAHDFox_AA
MHHHHHHPSTLNFQSETPTVQGEQDPSLVTLEHYGLQPPSAQQKLTPLDMNMPRLYGIRLILCFPTNPGMDKRQIYENLKKGLAHTVTSVPWISGHIGPEEGQDPKTRKVQILDSPYGFRFPYKDLTDALPPYAELQERNFPLAEFTTAQVGPIDVMPQGPNQPVFAAQANFVKGGLLLTVGVHHSACDALALDAILSTWSHNTAVASGGSGSFSTLDGPSNDRSPLMEGDLGNADVAAFPEYVLMPTPHSTEGDLSSMSGFQMPPLASRLFHFSPESLRKLKAEAGAFSSHDALCAFIWQRMTLARMHSGIFNDPPGDLTSRFCFAVNIRNRMSPPLPPSYMGNASMGCVTEKISVASMISNNGLKQASVTIRRSLNDFNSPRRATSTIGLLRSRPDPTDFKLSFNGFLGPDVVESSWADLGVYGHQWGDAIGTLDAVRIPGEGSDGTMMILPRLKDGGLDVVVGLSTAAMERLLEDEKFVSVAHS

SEQ ID NO: 35 - DNA, 1410 bp, Vasconcellea pubescens
  misc_feature 1..1410: note = VpAAT1
  source 1..1410: mol_type = genomic DNA
atgcatcatcaccaccaccatgctgaaaaggcctcttctctgatgttcaacgttagaaggcacgagccagagttgatcactccagctaaacctactccaagagagatcaagttgctgtccgacattgatgaccaggacggtttgagattccaggtcccaattatccagttctacaagaacaactcctccatgcagggtaagaacccagccaagattatcaagtctgctttggccgagactctggtccattactatccattggctggtagactgagagagggtttcggtagaaagttgatggttgagtgtaccggtgagggtatcttgttcattgaagctgatgccgacgttaccttgcacgaatttggtgatgatctgccaccaccattcccatgtttggtcgagttgttgtacgacgttccaggttcctccggtattatcgacactccattgctgttgatccaggtcaccagattgaagtgcggtggtttcatcttcgccttgagattgaaccacactatgtctgacgcttccggtttggttcagttcatgactgctgttggtgagatggctagaggtcaaagatccttgtccattcagccagtttgggagagacacttgttgaacgctagagatccaccaagagttacccacattcaccacgaatacgatgacttggaggacaccaagggtactatcattccattggacgacatggtccacaggtccttttttttcggtccatccgaaatggccgccatcagaagattggttccagctcactttcacagatccactacctccgaagttttgaccgcttacttgtggcgttgttacactattgccttgcaaccagacccagaggaagagatgagagttatctgtgtcgtcaactccaggaccaagttgaacccaccattgccaactggtttctacggtaacggtattgctttcccagctgctatctcccaggctaagaagatttgcgaaaacccattcggttacaccctgcagttggttaagcagaccaaggttgacgttaccgaagagtacatgagatccgctgctgacttgatggctatgaagggtagaccacacttcaccgtcgttagaaggtacatggtttccgacgttactagagccggtttcggtttggttgatttcggttggggtagaccagaaccagtttatggtggtccagctaagggtggtgttggtccaattccaggtgttacctcattcttcgtcccattcaagaacagaaagggtgagaagggtatcgttgtcccaacttgtttgccaactccagccatggaaagattcgccaagttgatgaacgagatcctgcagaaccagttgttggtttccgctgaagagaacaagtccgtgttcatcgtttccgctatctaa

SEQ ID NO: 36 - protein, 469 aa, Vasconcellea pubescens
  REGION 1..469: note = VpAAT1_AA
MHHHHHHAEKASSLMFNVRRHEPELITPAKPTPREIKLLSDIDDQDGLRFQVPIIQFYKNNSSMQGKNPAKIIKSALAETLVHYYPLAGRLREGFGRKLMVECTGEGILFIEADADVTLHEFGDDLPPPFPCLVELLYDVPGSSGIIDTPLLLIQVTRLKCGGFIFALRLNHTMSDASGLVQFMTAVGEMARGQRSLSIQPVWERHLLNARDPPRVTHIHHEYDDLEDTKGTIIPLDDMVHRSFFFGPSEMAAIRRLVPAHFHRSTTSEVLTAYLWRCYTIALQPDPEEEMRVICVVNSRTKLNPPLPTGFYGNGIAFPAAISQAKKICENPFGYTLQLVKQTKVDVTEEYMRSAADLMAMKGRPHFTVVRRYMVSDVTRAGFGLVDFGWGRPEPVYGGPAKGGVGPIPGVTSFFVPFKNRKGEKGIVVPTCLPTPAMERFAKLMNEILQNQLLVSAEENKSVFIVSAI

SEQ ID NO: 37 - DNA, 1368 bp, Vitis labrusca
  misc_feature 1..1368: note = AMAT
  source 1..1368: mol_type = genomic DNA
atgcatcatcaccaccatcacgcttctccatcttccccattggttttctccgttaacagatgcgttccccagatcgttagaccagctaacccaactccaagagaggttaagcagttgtccgacattgacgaccaagagggtagaagattccagatcccagtcatcatgttctacagaaacaaccccctgatggaaggtaaggacccagttaaggttatcagagaggctttgggtaaggccctggtttactactacccattcgctggtagattgatcgagggtgacaacagaaagttgatggttgactgtaccggtgagggtgtcttgttcattgaagctgatgctgacaccaccttggagaacttgggtgatgctattcagccaatgtgtccatgcttcgaggaattgctgtacgacgttccaggttccactactattttgggttccccattgatcctgatccaggtcaccagattgagatgcggtggtttcatcttcgccttgagattgaaccacactatgtctgacgctgccggtttgattcagttcttggacactattggtgagatggcccaaggtttgtctgtcccatctttgttgccaatctggcagagagagttgctgaacgctagaaacccaccaagaatcaccagaatccaccacgaatacgagaaggtcactaacaccaagggtactctgatggctatggacgaaaactccttggtccacaggtcatttttcttcggtagagaagagatcagggccctgcgtaatagattgccagcttctttgggtgcttgttccaccttcgaagttttgatggcctgtgtttggagatgcagaactatcgctttcgctgttgacccagacgaggttgttagaatctcctgcatcatcaacatgagaggtaagcacggtttcgagttgccaccaggttactacggtaacgcttttgttactccagcctccatcactaaggccggtatgttgtgtaagaacccattggagttcgccatcagactggtcaagaaagctaaggctgaaatgtcccaagagtacatcaagtccgttgccgacttgatggtcatcaagggtagacctttgttcacccagccaggtaacttcactgtttccgacgttactagagctggtttgggtgaagttgatttcggttggggtaagccagtttacggtggtgttgctagagcttgtccaatcatctccttcagaatgctgttcagaaactccaagggtgaagagggttccgttatcccaatttggttgccaccaccagtcatggaaagattcgagcaagagctgaagagaatgaccaagaaggccgagttgttgatcacctccatgttgtaa

SEQ ID NO: 38 - protein, 455 aa, Vitis labrusca
  REGION 1..455: note = AMAT_AA
MHHHHHHASPSSPLVFSVNRCVPQIVRPANPTPREVKQLSDIDDQEGRRFQIPVIMFYRNNPLMEGKDPVKVIREALGKALVYYYPFAGRLIEGDNRKLMVDCTGEGVLFIEADADTTLENLGDAIQPMCPCFEELLYDVPGSTTILGSPLILIQVTRLRCGGFIFALRLNHTMSDAAGLIQFLDTIGEMAQGLSVPSLLPIWQRELLNARNPPRITRIHHEYEKVTNTKGTLMAMDENSLVHRSFFFGREEIRALRNRLPASLGACSTFEVLMACVWRCRTIAFAVDPDEVVRISCIINMRGKHGFELPPGYYGNAFVTPASITKAGMLCKNPLEFAIRLVKKAKAEMSQEYIKSVADLMVIKGRPLFTQPGNFTVSDVTRAGLGEVDFGWGKPVYGGVARACPIISFRMLFRNSKGEEGSVIPIWLPPPVMERFEQELKRMTKKAELLITSML

SEQ ID NO: 39 - DNA, 1341 bp, Fragaria X ananassa
  misc_feature 1..1341: note = Pun1
  source 1..1341: mol_type = genomic DNA
atgcatcatcaccaccaccacgcttttgctttgccatcttctttggtttccgtctgcaacaagtcctttatcaagccatcctccttgaccccatccaccttgagatttcacaagctgtccttcatcgaccagtccctgtccaacatgtacatcccatgtgcattcttctaccccaaggtccaacaaagattggaggactctaagaactccgacgagttgtctcacattgcccacttgttgcaaacttccctgtcccagactctggtttcctactacccatacgctggtaagttgaaggacaacgctaccgttgactgtaacgacatgggtgctgagttcttgtccgtcagaatcaagtgttccatgtccgagattttggaccacccacatgcttctctggctgagtctatcgttttgccaaaggatttgccatgggccaacaactgtgaaggtggtaacttgttggttgtccaggtgtccaagttcgactgtggtggtattgctatctccgtttgcttctcccacaagatcggtgacggttgttccttgttgaacttcttgaacgactggtcctccgtcactagagatcacactactaccactttggtcccatccccaagattcgttggtgactctgttttctccacccagaagtacggttccttgatcactccacagatcctgtctgacttgaaccagtgtgtccagaagagactgatcttcccaactgacaagttggacgctttgagagctaaggttgctgaagagtccggtgttaagaacccaactagagctgaagttgtctccgccttgttgttcaagtgtgctactaaggcttcctcctccatgttgccatctaagttggtccactttctgaacatcaggaccatgatcaagccaagattgccaagaaacgccatcggtaacttgtcctccattttctccattgaggccaccaacatgcaggacatggaattgccaaccttggtcagaaacctgagaaaagaggttgaggtcgcctacaagaaggaccaagttgagcagaacgagctgatcttggaagttgtcgaatccatgagagagggtaagctgccattcgaaaacatggacggttacaagaacgtctacacctgttccaacctgtgcaagtacccttactacaccgttgatttcggttggggtagaccagagagagtttgtttgggtaacggtccatccaagaacgcattctttctgaaggactacaaggccggtcaaggtgttgaagccagagttatgttgcacaagcaacagatgtccgagttcgagagaaacgaagagttggtcgagttcattgcttaa

SEQ ID NO: 40 - protein, 446 aa, Fragaria X ananassa
  REGION 1..446: note = Pun1_AA
MHHHHHHAFALPSSLVSVCNKSFIKPSSLTPSTLRFHKLSFIDQSLSNMYIPCAFFYPKVQQRLEDSKNSDELSHIAHLLQTSLSQTLVSYYPYAGKLKDNATVDCNDMGAEFLSVRIKCSMSEILDHPHASLAESIVLPKDLPWANNCEGGNLLVVQVSKFDCGGIAISVCFSHKIGDGCSLLNFLNDWSSVTRDHTTTTLVPSPRFVGDSVFSTQKYGSLITPQILSDLNQCVQKRLIFPTDKLDALRAKVAEESGVKNPTRAEVVSALLFKCATKASSSMLPSKLVHFLNIRTMIKPRLPRNAIGNLSSIFSIEATNMQDMELPTLVRNLRKEVEVAYKKDQVEQNELILEVVESMREGKLPFENMDGYKNVYTCSNLCKYPYYTVDFGWGRPERVCLGNGPSKNAFFLKDYKAGQGVEARVMLHKQQMSEFERNEELVEFIA

SEQ ID NO: 41 - DNA, 1401 bp, Fragaria X ananassa
  misc_feature 1..1401: note = Dv3MaT
  source 1..1401: mol_type = genomic DNA
atgcatcatcaccaccaccacgacaacatcccaaacttgactattttggagcactccagaatctccccaccaccatctactattggtcacagatccttgcctctgaccttcttcgatatcgcctggttgttgtttccaccagtccaccacttgtacttctaccacttcccatactccaagtcccacttcaccgagactgttatccctaacttgaagcactccttgtccatcaccttgcagcactactttccattcgtcggtaagctgatcgtctacccaaacccacacgactctactagaaagccagagatcagacacgttgagggtgactctgttgctttgactttcgctgagactaccctggacttcaacgacttgtctgctaaccacccaagaaagtgcgagaacttctacccattggttccaccattgggtaacgctgtcaaagagtccgactacgttaccttgccagttttctccgttcaggtcacctacttcccaaactccggtatttccatcggtttgactaaccaccactctttgtccgacgctaacaccagattcggtttcttgaaggcttgggcttccgtttgtgaaactggtgaggatcagccattcctgaagaacggttctccaccagttttcgacagagttgttgtcaacccacagctgtacgagaacagattgaaccagaccagactgggtactttctaccaagctccttccttggttggttcctcatccgatagagttagagccactttcgttttggccagaactcacatctccggtttgaagaagcaggtcttgactcagttgccaatgttggagtacacctcttccttcaccgttacctgtggttacatctggtcctgtatcgtcaagtccttggtcaacatgggtgagaagaagggtgaggacgaattggagcaattcatcgtttccgttggttgcagatccagattggatccacctttgccagagaactacttcggtaactgttccgccccatgtatcgtcactatcaagaacggtgttctgaagggtgagaacggtttcgttatggctgctaagttgatcggtgagggtatctccaagatggtcaacaagaagggtggtatcttggagtacgctgacagatggtacgacggtttcaagatcccagctagaaagatgggtatctccggtactccaaagctgaacttctacgacattgacttcggttggggtaaggccatgaagtacgaggttgtttctatcgactactccgcctctgtttccttgtccgcttgtaaagaatccgctcaggacttcgagatcggtgtttgtttcccatccatgcagatggaagccttcggtaagattttcaacgacggtttggagtccgctatcgcttcttaa

SEQ ID NO: 42 - protein, 466 aa, Fragaria X ananassa
  REGION 1..466: note = Dv3MaT_AA
MHHHHHHDNIPNLTILEHSRISPPPSTIGHRSLPLTFFDIAWLLFPPVHHLYFYHFPYSKSHFTETVIPNLKHSLSITLQHYFPFVGKLIVYPNPHDSTRKPEIRHVEGDSVALTFAETTLDFNDLSANHPRKCENFYPLVPPLGNAVKESDYVTLPVFSVQVTYFPNSGISIGLTNHHSLSDANTRFGFLKAWASVCETGEDQPFLKNGSPPVFDRVVVNPQLYENRLNQTRLGTFYQAPSLVGSSSDRVRATFVLARTHISGLKKQVLTQLPMLEYTSSFTVTCGYIWSCIVKSLVNMGEKKGEDELEQFIVSVGCRSRLDPPLPENYFGNCSAPCIVTIKNGVLKGENGFVMAAKLIGEGISKMVNKKGGILEYADRWYDGFKIPARKMGISGTPKLNFYDIDFGWGKAMKYEVVSIDYSASVSLSACKESAQDFEIGVCFPSMQMEAFGKIFNDGLESAIAS

SEQ ID NO: 43 - DNA, 1326 bp, Fragaria X ananassa
  misc_feature 1..1326: note = NtHCT
  source 1..1326: mol_type = genomic DNA
atgcatcatcaccaccaccacaagatcgaggtcaaagaatccaccatggttaagccagctgctgagactccacaacagagattgtggaactccaacgttgacttggtcgtcccaaacttccacactccatccgtctacttctacagaccaactggttccccaaacttcttcgacggtaaggttttgaaagaggccttgtccaaggctctggttccattttatccaatggccggtagactgtgcagagatgaggatggtagaatcgagatcgactgtaagggtcagggtgttttgttcgttgaagctgaatccgacggtgttgttgatgacttcggtgatttcgctccaaccttggagttgagacagttgattccagctgttgactactcccagggtattcagtcttacgccttgttggtcttgcagatcacccactttaagtgtggtggtgtttccttgggtgttggtatgcaacatcatgctgctgatggtgcttccggtctgcactttattaacacttggtccgacatggccagaggtttggacttgactattccaccattcatcgacagaaccctgctgagagctagagatccaccacaaccacaattcccacacgttgaataccaaccaccaccaaccttgaaggttactccagagaacactccaatctccgaagctgttccagaaacctccgtttccatcttcaagctgaccagagatcagatcaacaccttgaaggccaagtccaaagaggacggtaataccgttaactactcctcctacgagatgttggctggtcacgtttggagatccacttgtatggctagaggattggctcacgaccaagagactaagttgtacattgctaccgacggtagatccagattgaggccatctttgccaccaggttacttcggtaacgttatcttcactactaccccaatcgctgttgctggtgacattcagtctaagccaatttggtacgctgcctccaagttgcatgatgctttggctagaatggacaacgactacttgagatccgccttggactacttggaattgcagccagatttgaaggccttggttagaggtgctcacaccttcaagtgtccaaacttgggtattacctcctggtccagattgccaattcacgatgctgatttcggttggggtagaccaattttcatgggtccaggtggtattgcctacgagggtttgtctttcattctgccatctccaactaacgacggttcccagtctgttgctatttccttgcaagctgagcacatgaagctgttcgagaagttcctgtacgacttctaa

SEQ ID NO: 44 - protein, 441 aa, Fragaria X ananassa
  REGION 1..441: note = NtHCT_AA
MHHHHHHKIEVKESTMVKPAAETPQQRLWNSNVDLVVPNFHTPSVYFYRPTGSPNFFDGKVLKEALSKALVPFYPMAGRLCRDEDGRIEIDCKGQGVLFVEAESDGVVDDFGDFAPTLELRQLIPAVDYSQGIQSYALLVLQITHFKCGGVSLGVGMQHHAADGASGLHFINTWSDMARGLDLTIPPFIDRTLLRARDPPQPQFPHVEYQPPPTLKVTPENTPISEAVPETSVSIFKLTRDQINTLKAKSKEDGNTVNYSSYEMLAGHVWRSTCMARGLAHDQETKLYIATDGRSRLRPSLPPGYFGNVIFTTTPIAVAGDIQSKPIWYAASKLHDALARMDNDYLRSALDYLELQPDLKALVRGAHTFKCPNLGITSWSRLPIHDADFGWGRPIFMGPGGIAYEGLSFILPSPTNDGSQSVAISLQAEHMKLFEKFLYDF

SEQ ID NO: 45 - DNA, 1341 bp, Fragaria X ananassa
  misc_feature 1..1341: note = DBATAca
  source 1..1341: mol_type = genomic DNA
atgcatcatcaccatcaccacgctggttctaccgagttcgttgttagatccttggagagagttatggttgctccatctcaaccatccccaaaggctttcttgcagttgtccactttggacaacttgccaggtgtcagagagaacatcttcaacaccttgttggtctacaacgcctccgacagagtttctgttgatccagccaaggttatcagacaggccttgtccaaggttctggtttactactctccattcgccggtagactgagaaagaaagaaaacggtgacttggaggttgagtgtactggtgaaggtgctttgttcgttgaagctatggctgacactgacctgtctgttttcggtgatttggacgactactccccatctttggagcagttgttgttctgtttgccaccagacactgacatcgaggacattcacccattggttgttcaggtcaccagattcacttgtggtggtttcgttgttggtgtctccttctgtcacggtatctgtgatggtttgggtgctggtcagttcttgattgctgttggtgaaatggccagaggtgagattaagccatcttccgagccaatctggaagagagagttgttgaagccagaggacccactgtacagattgcagtactaccacttccagttgatctgtccaccatccaccttcggtaagatcgttcaaggttccttggttatcacctccgagactatcaactgcatcaagcagtgcttgagagaagagtccaaagagttctgttccgccttcgaagttgtttccgctttggcttggatcgctagaactagagccttgcagattccacacaacgagaacgtcaagctgatcttcgctatggacatgagaaagctgttcaacccaccactgtccaagggttactacggtaacttcgttggtactgtttgcgccatggacaacgtcaaggatttgttgtctggttccttgctgagagtcgtcagaatcatcaagaaggccaaggtttccctgaacgagcacttcacttccactatcgttactccaagatctggttccgacgagtccatcaactacgagaacatcgttggtttcggtgacagacgtagattgggtttcgacgaagttgatttcggttggggtcacgctgacaacgtttctttggttcaacacggtctgaaggacgtttccgttgttcagtcctacctgctgttcatcagaccaccaaagaacaacccagacggtatcaagatcctgtcctttatgccaccaccaatcgtcaagtccttcaagttcgagatggaaaccatgaccaacaagtacgtcaccaagccttaa

SEQ ID NO: 46 - protein, 446 aa, Fragaria X ananassa
  REGION 1..446: note = DBATAca_AA
MHHHHHHAGSTEFVVRSLERVMVAPSQPSPKAFLQLSTLDNLPGVRENIFNTLLVYNASDRVSVDPAKVIRQALSKVLVYYSPFAGRLRKKENGDLEVECTGEGALFVEAMADTDLSVFGDLDDYSPSLEQLLFCLPPDTDIEDIHPLVVQVTRFTCGGFVVGVSFCHGICDGLGAGQFLIAVGEMARGEIKPSSEPIWKRELLKPEDPLYRLQYYHFQLICPPSTFGKIVQGSLVITSETINCIKQCLREESKEFCSAFEVVSALAWIARTRALQIPHNENVKLIFAMDMRKLFNPPLSKGYYGNFVGTVCAMDNVKDLLSGSLLRVVRIIKKAKVSLNEHFTSTIVTPRSGSDESINYENIVGFGDRRRLGFDEVDFGWGHADNVSLVQHGLKDVSVVQSYLLFIRPPKNNPDGIKILSFMPPPIVKSFKFEMETMTNKYVTKP

SEQ ID NO: 47 - DNA, 1377 bp, Streptomyces gancidicus
  misc_feature 1..1377: note = TSga
  source 1..1377: mol_type = genomic DNA
atgcatcatcaccaccaccacaagtctggttctgctgctggtgatgctggtagaaccgttttggttagatctggtgaagcttccggtgagagagttagattgtccgtttacgacctggtcaacggtactttcggttcctccagaaccttctactacagacagagattggacaccgaggctttgagagagtccttgagaagaactttggtccactaccctttgctgaccggtagattggttagagatgctgacagaggtttgtccgttgtttgtgatgatgctggtgctgttttcgctgaaactgactctgatagaccaatgccagattacggtccagaccacagagttggtgatgacttgagaaggtacatccacccagttaacgccttcagagttgttggtcatgacacccctttgttgaccgttaaggttacccatatgagaggtggtggttccgttttgggtgtttccactaaccactctgttgttgacggttccggttgcttggatttcttgttgcactggtccagaacccacagaggtttggatcatagagccccatctcacgacagagctttgttggatggtttggctgctggtgttccaccagctccagatgattctcagtacgctgttatcactggtagagccaagttcggtttcatctggcgtgttaacgctagagccagaagagttagaacctttaccgtcagattctcctcagccgaggttttggcattgagagaaactgctagagctggtggtgatcacgttagagctacttccggtgatgctttgtctgcccacatttggagagttttgggtgccgttagagacagagaaccagctgctactgaaagattgggtatcgtcgttggtttgagaggtccattgtctgaacatctgccacatggttacggtggtaacgctgtttccaacatcactgctgctttgccagctagagccttgagagaagaaccattggctcatactgcttccgctgttagagaagccttggacagagttactccagagagaatcagagaagaggctgctttcttggaggctcaaagaagggctggtagagtcaacagagtcttgtccagaatggctttggactccttcgctgacactgtttccttgaacaacgtttccagattgcccgtctacgctattgagtttggtgctggtagaccattctggttcgaacatccagctactccagttccatggaccgttttgattactccaactccagatgacgaccactccagagatgttcacttgtctgttccaagagaagctgctgaggcattgagaactccagaatggtccagaagattgcacctgagagaatcttccccagacagattttaa

SEQ ID NO: 48 - protein, 458 aa, Streptomyces gancidicus
  REGION 1..458: note = Tsga_AA
MHHHHHHKSGSAAGDAGRTVLVRSGEASGERVRLSVYDLVNGTFGSSRTFYYRQRLDTEALRESLRRTLVHYPLLTGRLVRDADRGLSVVCDDAGAVFAETDSDRPMPDYGPDHRVGDDLRRYIHPVNAFRVVGHDTPLLTVKVTHMRGGGSVLGVSTNHSVVDGSGCLDFLLHWSRTHRGLDHRAPSHDRALLDGLAAGVPPAPDDSQYAVITGRAKFGFIWRVNARARRVRTFTVRFSSAEVLALRETARAGGDHVRATSGDALSAHIWRVLGAVRDREPAATERLGIVVGLRGPLSEHLPHGYGGNAVSNITAALPARALREEPLAHTASAVREALDRVTPERIREEAAFLEAQRRAGRVNRVLSRMALDSFADTVSLNNVSRLPVYAIEFGAGRPFWFEHPATPVPWTVLITPTPDDDHSRDVHLSVPREAAEALRTPEWSRRLHLRESSPDRF

SEQ ID NO: 49 - DNA, 1428 bp, Streptomyces violaceusniger
  misc_feature 1..1428: note = TSvi
  source 1..1428: mol_type = genomic DNA
atgcatcatcaccatcaccacactgcttccgctactgaatctggtgctaagagaaccttcactgttagagctggtgaagcttccggtgacagattgagattgtccgtctacgacatgctgatcggtccaatctacactccaagagctttcttctaccgtgaaaccttggacggtgaagctttgagagcttccttgaccagaaccctgagaaacttcccaatcctgtccggtagaatgaagagggattctgacggtggtttgtccgttttgtgtgatgacggtggtgttagattcgttgaggcttacgcttctgagccaatgccagattacggtccaagacacactgctaagaagggtttggaaagacacttgtcccacgctatgccattctgggttgttgatcatgacaccccactgttcaccgttaagttgactcatatgaagggtggtggttccatcttgggtttgactatgaaccacgctgttgctgacggttcttcctacatgtctttcttggagtcctgggtcaacgagcatagaggtttgggttacgctaagccatctcacgacagaggtgttatcgatactttgggtgctttggctactggtgacactagaactggtggtgctcacttgactgttactggtagaggtcaaaaggctgccttcatcggtagaactgttatgggttccttgggtaacgttactaccgtcactactagattcactgctactgagttggccaccatgaaggatactgctatggctgatttggccggtactgaaagatgggtttccactaacgatgctttgactgcccacttgtggaaggttttgggtgagttgagagatagaccagacgcttccgaagagagattgggtttgattgctgacttcagatcttccgctggtgaggctgttccagatgattactggggtaacgctgttaccaacaccagaccaggtatgactgctgctgaattgagatccagaccattgggtgaagttgctgctgctgttagagcaggtcatgctatgaacaccgaagagagaatcagagaagagactgccttcttgtgtgctgagagagatgctggtagattcaagagggttatgaccactatggctttggacgctttcgacggtactatcgctattaacaactggtccaagctgcccttctacagaattgactttggtcagggtgctccattctggtacgacttcacttctactccaatcccatccaccgttcacattgctccaactccagctgatcaaaacggtgccagagatgttcatatggccttgccaagaactcaggtcagagcattgagagaaccatcttgggcttccagattccacagatacgctgaatccggtgagactttcccattgactttcatggactccaaggccaagagatag

SEQ ID NO: 50 - protein, 475 aa, Streptomyces violaceusniger
  REGION 1..475: note = Tsvi_AA
MHHHHHHTASATESGAKRTFTVRAGEASGDRLRLSVYDMLIGPIYTPRAFFYRETLDGEALRASLTRTLRNFPILSGRMKRDSDGGLSVLCDDGGVRFVEAYASEPMPDYGPRHTAKKGLERHLSHAMPFWVVDHDTPLFTVKLTHMKGGGSILGLTMNHAVADGSSYMSFLESWVNEHRGLGYAKPSHDRGVIDTLGALATGDTRTGGAHLTVTGRGQKAAFIGRTVMGSLGNVTTVTTRFTATELATMKDTAMADLAGTERWVSTNDALTAHLWKVLGELRDRPDASEERLGLIADFRSSAGEAVPDDYWGNAVTNTRPGMTAAELRSRPLGEVAAAVRAGHAMNTEERIREETAFLCAERDAGRFKRVMTTMALDAFDGTIAINNWSKLPFYRIDFGQGAPFWYDFTSTPIPSTVHIAPTPADQNGARDVHMALPRTQVRALREPSWASRFHRYAESGETFPLTFMDSKAKR

SEQ ID NO: 51 - DNA, 672 bp, Shigella sonnei
  misc_feature 1..672: note = CAT
  source 1..672: mol_type = genomic DNA
atgcatcatcaccatcaccacgagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgaaggggcggcctga

SEQ ID NO: 52 - protein, 223 aa, Shigella sonnei
  REGION 1..223: note = CAT_AA
MHHHHHHEKKITGYTTVDISQWHRKEHFEAFQSVAQCTYNQTVQLDITAFLKTVKKNKHKFYPAFIHILARLMNAHPELRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEGAA

SEQ ID NO: 53 - DNA, 1374 bp, Saccharomyces cerevisiae
  misc_feature 1..1374: note = EHT
  source 1..1374: mol_type = genomic DNA
atgcatcatcaccatcaccactcagaagtttccaaatggccagcaatcaacccattccattggggatacaatggtacagtttcgcatattgtcggtgaaaatggttccattaaactccatttaaaagacaacaaggagcaagttgattttgacgagttcgctaacaaatatgtcccaacgttgaagaatggtgcccaattcaaattgagtccttacttgttcacaggtattttgcaaactttgtacttaggtgctgctgatttctctaagaaatttcctgtattctacggcagggaaattgtcaaattctcggatggtggagtttgcaccgctgactggctcatagattcatggaaaaaggattatgagttcgatcaaagtactacgagctttgataaaaaaaaatttgataaagacgagaaggcgacacatccagaaggatggcctcgtttacaaccacgtacaaggtacctgaaagataatgagttggaagaactacgggaggttgatctacccctagtagttattctacatggtcttgctggtggtagtcatgagccgattataagatctcttgctgaaaacctgtctcgcagtgggagatttcaagtggtcgtcctaaataccagaggttgtgcacgttccaaaattaccaccagaaatttatttacagcttatcacacaatggatattcgcgagtttttgcaaagagaaaagcaaagacatccagatagaaaactatacgctgtgggatgctcttttggtgctacgatgctggcaaactatctgggagaagagggcgataaatcacctttatccgcagctgctactttgtgcaatccttgggatcttctcctttcagcaattaggatgagccaggattggtggtcaagaactttattttccaaaaatattgcgcaattcttaacaagaaccgttcaggttaatatgggtgaattaggagttccaaatggctctctccccgatcatcctcccacagtcaagaatccatctttctatatgttcacgcctgaaaatctaataaaggcaaagagctttaaatcgacccgggaatttgatgaagtgtacactgcgcctgctttaggcttcccaaatgctatggagtattataaagcggccagctcaataaacagagttgatacaattcgggttcctacccttgttatcaattccagggatgatcctgttgtcggcccagatcaaccatactcaatcgtggaaaagaatcctcgtattttgtattgtagaaccgatttaggtggtcatttagcttacctagataaagacaacaactcgtgggctaccaaggcaattgcagaatttttcactaagtttgatgaattagtcgtatga

SEQ ID NO: 54 - protein, 457 aa, Saccharomyces cerevisiae
  REGION 1..457: note = EHT_AA
MHHHHHHSEVSKWPAINPFHWGYNGTVSHIVGENGSIKLHLKDNKEQVDFDEFANKYVPTLKNGAQFKLSPYLFTGILQTLYLGAADFSKKFPVFYGREIVKFSDGGVCTADWLIDSWKKDYEFDQSTTSFDKKKFDKDEKATHPEGWPRLQPRTRYLKDNELEELREVDLPLVVILHGLAGGSHEPIIRSLAENLSRSGRFQVVVLNTRGCARSKITTRNLFTAYHTMDIREFLQREKQRHPDRKLYAVGCSFGATMLANYLGEEGDKSPLSAAATLCNPWDLLLSAIRMSQDWWSRTLFSKNIAQFLTRTVQVNMGELGVPNGSLPDHPPTVKNPSFYMFTPENLIKAKSFKSTREFDEVYTAPALGFPNAMEYYKAASSINRVDTIRVPTLVINSRDDPVVGPDQPYSIVEKNPRILYCRTDLGGHLAYLDKDNNSWATKAIAEFFTKFDELVV

SEQ ID NO: 55 - DNA, 1596 bp, Saccharomyces cerevisiae
  misc_feature 1..1596: note = ATF
  source 1..1596: mol_type = genomic DNA
atgcatcatcaccatcaccacaatgaaatcgatgagaaaaatcaggcccccgtgcaacaagaatgcctgaaagagatgattcagaatgggcatgctcggcgtatgggatctgttgaagatctgtatgttgctctcaacagacaaaacttatatcgaaacttctgcacgtatggagaattgagtgattactgtactagggatcagctcacattagctttgagggaaatctgcctgaaaaatccaactcttttacatattgttctaccaacaagatggccaaatcatgaaaattattatcgcagttccgaatactattcacggccacatccagtgcatgattatatttcagtattacaagaattgaaactgagtggtgtggttctcaatgaacaacctgagtacagtgcagtaatgaagcaaatattagaagagttcaaaaatagtaagggttcctatactgcaaaaatttttaaacttactaccactttgactattccttactttggaccaacaggaccgagttggcggctaatttgtcttccagaagagcacacagaaaagtggaaaaaatttatctttgtatctaatcattgcatgtctgatggtcggtcttcgatccacttttttcatgatttaagagacgaattaaataatattaaaactccaccaaaaaaattagattacattttcaagtacgaggaggattaccaattattgaggaaacttccagaaccgatcgaaaaggtgatagactttagaccaccgtacttgtttattccgaagtcacttctttcgggtttcatctacaatcatttgagattttcttcaaaaggtgtctgtatgagaatggatgatgtggaaaaaaccgatgatgttgtcaccgagatcatcaatatttcaccaacagaatttcaagcgattaaagcaaatattaaatcaaatatccaaggtaagtgtactatcactccgtttttacatgtttgttggtttgtatctcttcataaatggggtaaatttttcaaaccattgaacttcgaatggcttacggatatttttatccccgcagattgccgctcacaactaccagatgatgatgaaatgagacagatgtacagatatggcgctaacgttggatttattgacttcaccccctggataagcgaatttgacatgaatgataacaaagaaaatttttggccacttattgagcactaccatgaagtaatttcggaagctttaagaaataaaaagcatctccatggcttagggttcaatatacaaggcttcgttcaaaaatatgtgaacattgacaaggtaatgtgcgatcgtgccatcgggaaaagacgcggaggtacattgttaagcaatgtaggtctgtttaatcagttagaggagcccgatgccaaatattctatatgcgatttggcatttggccaatttcaaggatcctggcaccaagcattttccttgggtgtttgttcgactaatgtaaaggggatgaatattgttgttgcttcaacaaagaatgttgttggtagtcaagaatctctcgaagagctttgctccatttacaaagctctccttttaggcccttag

SEQ ID NO: 56 - protein, 531 aa, Saccharomyces cerevisiae
  REGION 1..531: note = ATF_AA
MHHHHHHNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 57 - DNA, 1344 bp, Arabidopsis thaliana
  misc_feature 1..1344: note = Aco_op
  source 1..1344: mol_type = genomic DNA
atgcatcaccatcaccatcacactagtggatccatggcagtcctatcctcagctgatagggctagtaacgaaaagaaggtaaagtcatcttacttcgacttgcctccaatggaaatgtcagttgcattcccacaagccacaccagcttctacgtttcccccgtgcacttctgattactatcactttaatgacttgttgacaccagaagagcaggcaattagaaagaaggtaagagagtgtatggaaaaagaagttgctccgattatgactgaatactgggagaaggcagagtttccatttcatataacacctaagctaggggctatgggagttgcaggcggatctatcaaaggttacggttgtccaggcctaagcatcacagccaatgctatcgcaacagccgaaattgcaagggttgatgccagttgttctacgttcattttagtccatagttctttaggaatgctgacaattgctttatgcggtagtgaagcacaaaaagagaaataccttccatccttggcacaacttaatacagtggcctgctgggcgcttactgagccagataatggttctgatgcttcaggattgggaaccacagcgactaaggtggaaggcggttggaagattaacggtcaaaaaaggtggataggaaactcaacattcgcggatttattgattatctttgctagaaacacgactaccaaccaaatcaacggcttcattgtaaagaaagatgctcctggcttaaaagcaaccaaaatccctaataaaattggtttgaggatggtacaaaacggggatatcttgttacagaacgtgtttgtgcccgacgaagatcgtctacccggtgttaattctttccaagacacttccaaggtattagcagtctcacgtgttatggtagcttggcagcctattggtatctctatgggtatctacgatatgtgtcatagatacctgaaagaaaggaagcagtttggagctcctttagctgcttttcaacttaaccagcaaaaattggtacaaatgttaggaaatgttcaagcgatgttccttatgggctggagattgtgtaagttatacgaaactggtcaaatgacacctggtcaggcgtcattaggtaaggcttggatatcctctaaggcaagagaaacagcaagtttgggcagagaacttttaggtggaaacggtattttagccgatttcctagttgcgaaagcattctgtgacttggagcctatatacacatacgaagggacttacgatattaatacattagtgacggggagagaagttacaggcattgctagtttcaaaccagctacaaggtctagattataa

SEQ ID NO: 58 - protein, 447 aa, Arabidopsis thaliana
  REGION 1..447: note = Aco_AA
MHHHHHHTSGSMAVLSSADRASNEKKVKSSYFDLPPMEMSVAFPQATPASTFPPCTSDYYHFNDLLTPEEQAIRKKVRECMEKEVAPIMTEYWEKAEFPFHITPKLGAMGVAGGSIKGYGCPGLSITANAIATAEIARVDASCSTFILVHSSLGMLTIALCGSEAQKEKYLPSLAQLNTVACWALTEPDNGSDASGLGTTATKVEGGWKINGQKRWIGNSTFADLLIIFARNTTTNQINGFIVKKDAPGLKATKIPNKIGLRMVQNGDILLQNVFVPDEDRLPGVNSFQDTSKVLAVSRVMVAWQPIGISMGIYDMCHRYLKERKQFGAPLAAFQLNQQKLVQMLGNVQAMFLMGWRLCKLYETGQMTPGQASLGKAWISSKARETASLGRELLGGNGILADFLVAKAFCDLEPIYTYEGTYDINTLVTGREVTGIASFKPATRSRL

SEQ ID NO: 59 - DNA, 1554 bp, Fragaria X ananassa
  misc_feature 1..1554: note = Pct_Me
  source 1..1554: mol_type = genomic DNA
atgagaaaggttgaaattattaccgctgaacaagctgctcaattggttaaggacaacgacaccattacctctattggtttcgtttcttctgctcacccagaagctttgaccaaggctttggaaaagagattcttggacaccaacaccccacaaaacttgacctacatttacgctggttctcaaggtaagagagacggtagagctgctgaacacttggctcacaccggtttgttgaagagagctattattggtcactggcaaaccgttccagctattggtaagttggctgttgaaaacaagattgaagcttacaacttctctcaaggtaccttggttcactggttcagagctttggctggtcacaagttgggtgttttcaccgacattggtttggaaaccttcttggacccaagacaattgggtggtaagttgaacgacgttaccaaggaagacttggttaagttgattgaagttgacggtcacgaacaattgttctacccaaccttcccagttaacgttgctttcttgagaggtacctacgctgacgaatctggtaacattactatggacgaagaaattggtccattcgaatctacctctgttgctcaagctgttcacaactgtggtggtaaggttgttgttcaagttaaggacgttgttgctcacggttctttggacccaagaatggttaagattccaggtatttacgttgactacgttgttgttgctgctccagaagaccaccaacaaacctacgactgtgaatacgacccatctttgtctggtgaacacagagctccagaaggtgctaccgacgctgctttgccaatgtctgctaagaagattattggtagaagaggtgctttggaattgaccgaaaacgctgttgttaacttgggtgttggtgctccagaatacgttgcttctgttgctggtgaagaaggtattgctgacaccattaccttgaccgttgaaggtggtgctattggtggtgttccacaaggtggtgctagattcggttcttcaagaaacgctgacgctattattgaccacacctaccaattcgacttctacgacggtggtggtttggacattgcttacttgggtttggctcaatgtgacggttctggtaacattaacgtttctaagttcggtaccaacgttgctggttgtggtggtttcccaaacatttctcaacaaaccccaaacgtttacttctgtggtaccttcaccgctggtggtttgaagattgctgttgaagacggtaaggttaagattttgcaagaaggtaaggctaagaagttcattaaggctgttgaccaaattaccttcaacggttcttacgctgctagaaacggtaagcacgttttgtacattaccgaaagatgtgttttcgaattgaccaaggaaggtttgaagttgattgaagttgctccaggtattgacattgaaaaggacattttggctcacatggacttcaagccaattattgacaacccaaagttgatggacgctagattgttccaagacggtccaatgggtttgaagaagtaa

SEQ ID NO: 60 - protein, 517 aa, Fragaria X ananassa
  REGION 1..517: note = Pct_Me_AA
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGATDAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKK

SEQ ID NO: 61 - DNA, 1629 bp, Cupriavidus necator
  misc_feature 1..1629: note = Pct_Cn
  source 1..1629: mol_type = genomic DNA
atgaaggttattaccgcaagagaagctgctgcattggttcaagacggttggaccgttgcttctgctggtttcgttggtgctggtcacgctgaagctgttaccgaagcattggaacaaagattcttgcaatctggattgccaagagacttgaccttggtttactctgctggacaaggtgacagaggtgctagaggtgttaatcacttcggtaacgctggtatgaccgcttctattgttggtggtcactggagatctgctaccagattggctaccttggctatggctgaacaatgtgaaggttacaacttgccacaaggtgttttgacccacctatacagagctattgctggtggtaagccaggtgttatgaccaagattggtttgcacaccttcgttgacccaagaaccgctcaagacgctagataccacggtggtgctgttaatgaaagagctagacaagctatagctgaaggtaaggcttgttgggttgacgctgttgacttcagaggtgacgaatacttgttctacccatctttcccaattcactgtgctttgattagatgtaccgctgctgacgctagaggtaacttgtctacccacagagaagcattccaccacgaattgttggctatggctcaagctgctcacaactctggtggtattgttattgctcaagttgaatctttggttgaccaccacgaaattttgcaagctattcacgttccaggtattttggttgactacgttgttgtttgtgacaacccagctaaccaccaaatgaccttcgctgaatcttacaacccagcttacgttaccccttggcaaggtgaagctgctgttgctgaagctgaagctgctccagttgcagctggtccattggacgctagaaccattgttcaaaggagagctgttatggaattggctagaagggctccaagagttgttaatttgggtgttggtatgccagctgctgttggtatgttggctcaccaagcaggattggacggtttcaccttgaccgttgaagctggtccaattggtggtaccccagctgacggtttgtctttcggagcttctgcttacccagaagctgttgttgaccaaccagctcaattcgacttctacgaaggtggtggtattgacttggctattttgggtttggctgaattggacggtcacggtaacgttaatgtttctaagttcggtgaaggtgaaggtgcttctatagctggtgttggtggtttcattaacattacccaatctgctagagctgttgttttcatgggtaccttgaccgctggtggattggaagttagagctggtgacggtggtttgcaaattgttagagaaggtagagttaagaagattgttcctgaagtttctcacttgtctttcaacggaccatacgttgcttctttgggtattccagttttgtacattaccgaaagagctgttttcgaaatgagagcaggtgctgacggtgaagctagattgaccttggttgaaattgctccaggtgttgacttgcaaagagacgttttggaccaatgttctaccccaattgctgtagctcaagacttgagagaaatggacgctagattgttccaagctggaccattgcacttgtaa

SEQ ID NO: 62 - protein, 542 aa, Cupriavidus necator
  REGION 1..542: note = Pct_CN_AA
MKVITAREAAALVQDGWTVASAGFVGAGHAEAVTEALEQRFLQSGLPRDLTLVYSAGQGDRGARGVNHFGNAGMTASIVGGHWRSATRLATLAMAEQCEGYNLPQGVLTHLYRAIAGGKPGVMTKIGLHTFVDPRTAQDARYHGGAVNERARQAIAEGKACWVDAVDFRGDEYLFYPSFPIHCALIRCTAADARGNLSTHREAFHHELLAMAQAAHNSGGIVIAQVESLVDHHEILQAIHVPGILVDYVVVCDNPANHQMTFAESYNPAYVTPWQGEAAVAEAEAAPVAAGPLDARTIVQRRAVMELARRAPRVVNLGVGMPAAVGMLAHQAGLDGFTLTVEAGPIGGTPADGLSFGASAYPEAVVDQPAQFDFYEGGGIDLAILGLAELDGHGNVNVSKFGEGEGASIAGVGGFINITQSARAVVFMGTLTAGGLEVRAGDGGLQIVREGRVKKIVPEVSHLSFNGPYVASLGIPVLYITERAVFEMRAGADGEARLTLVEIAPGVDLQRDVLDQCSTPIAVAQDLREMDARLFQAGPLHL

SEQ ID NO: 63 - DNA, 6360 bp, synthetic construct
  misc_feature 1..6360: note = pYP004
  source 1..6360: mol_type = other DNA
gagctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaagttaacatgcatcaccatcaccatcacactagtggatcccccgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctg

SEQ ID NO: 64 - DNA, 6907 bp, synthetic construct
  misc_feature 1..6907: note = pYP083
  source 1..6907: mol_type = other DNA
cagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgcggtgtgaaataccgcacagatgcgtaaggagaaaataccgcatgagctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaagaattcaaaacgatgcatcaccatcaccaccacgagactatgcagactatcgacttctcattccaggttagaaagtgtcagccagagttgatcgctccagctaacccaactccatacgagttcaagcaattgtccgacgttgacgaccaacagtccttgagattccagttgccattggttaacatctaccaccacaacccatccttggagggtagagatccagttaaggttatcaaagaggctatcgctaaggctttggttttctactacccattggctggtagattgagagagggtcctggtagaaagttgttcgttgagtgtactggtgagggtatcttgttcattgaagctgacgctgacgtttccttggagcagttcagagatactttgccatactccttgtcctccatggaaaacaacatcatccacaactcattgaactccgacggtgttttgaactcccctttgttgttgatccaggttactagattgaagtgtggtggtttcatcttcggtatccacttcgaccacactatggctgacggttttggtatcgctcagttcatgaaggctattgctgagatcgctagaggtgctttcgctccatctattttgccagtttggcagagagctttgttgactgctagagatcctccaagaatcactgttagacactacgagtacgaccaggttgttgacactaagtccactttgatcccagctaacaacatgatcgacagattgttcttcttcactcagagacagatctccacattgagacagactttgccagctcacttgcacgactgttcttcattcgaggttttgactgcttacgtttggagattgagaactatcgctttccagttgaagccagaggaagaggttagattcttgtgtgttgttaacttgagatccaagatcgacatcccattgggtttctacggtaacgctatcgttttcccagctgttatcactactgttgctaagttgtgtggtaaccctttgggttacgctgttgacttgatcagaaaggctaaggctaaagctacaaaagagtacatcaagtccatggttgacttcatggttatcaagggtagaccaagattcactgagatcggtccattcatgatgtccgacattactagaatcggtttcgagaacgttgacttcggttggggtaaggctattttcggtggtccaattatcggtggttgtggtatcatcagaggtatgatctcttactccattgctttcatgaacagaaacggtgagaagggaatcgttgttccattgtgtttgccaccaccagctatggaaagattcagagctaacgttcacgcttccttgcaggttatccaggttttggacaaggttgacagagacatgcaaacaatcttgtccgctttgtaaaggggcggccgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccgaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtg

SEQ ID NO: 65 - DNA, 8270 bp, synthetic construct
  misc_feature 1..8270: note = pYP096
  source 1..8270: mol_type = other DNA
acaggaaacagctatgaccatgattacgccaagcgcgcaattaaccctcactaaagggaacaaaagctggagctcaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacagttaacatgcatcaccatcaccatcacactagtggatccatgagaaaggttgaaattattaccgctgaacaagctgctcaattggttaaggacaacgacaccattacctctattggtttcgtttcttctgctcacccagaagctttgaccaaggctttggaaaagagattcttggacaccaacaccccacaaaacttgacctacatttacgctggttctcaaggtaagagagacggtagagctgctgaacacttggctcacaccggtttgttgaagagagctattattggtcactggcaaaccgttccagctattggtaagttggctgttgaaaacaagattgaagcttacaacttctctcaaggtaccttggttcactggttcagagctttggctggtcacaagttgggtgttttcaccgacattggtttggaaaccttcttggacccaagacaattgggtggtaagttgaacgacgttaccaaggaagacttggttaagttgattgaagttgacggtcacgaacaattgttctacccaaccttcccagttaacgttgctttcttgagaggtacctacgctgacgaatctggtaacattactatggacgaagaaattggtccattcgaatctacctctgttgctcaagctgttcacaactgtggtggtaaggttgttgttcaagttaaggacgttgttgctcacggttctttggacccaagaatggttaagattccaggtatttacgttgactacgttgttgttgctgctccagaagaccaccaacaaacctacgactgtgaatacgacccatctttgtctggtgaacacagagctccagaaggtgctaccgacgctgctttgccaatgtctgctaagaagattattggtagaagaggtgctttggaattgaccgaaaacgctgttgttaacttgggtgttggtgctccagaatacgttgcttctgttgctggtgaagaaggtattgctgacaccattaccttgaccgttgaaggtggtgctattggtggtgttccacaaggtggtgctagattcggttcttcaagaaacgctgacgctattattgaccacacctaccaattcgacttctacgacggtggtggtttggacattgcttacttgggtttggctcaatgtgacggttctggtaacattaacgtttctaagttcggtaccaacgttgctggttgtggtggtttcccaaacatttctcaacaaaccccaaacgtttacttctgtggtaccttcaccgctggtggtttgaagattgctgttgaagacggtaaggttaagattttgcaagaaggtaaggctaagaagttcattaaggctgttgaccaaattaccttcaacggttcttacgctgctagaaacggtaagcacgttttgtacattaccgaaagatgtgttttcgaattgaccaaggaaggtttgaagttgattgaagttgctccaggtattgacattgaaaaggacattttggctcacatggacttcaagccaattattgacaacccaaagttgatggacgctagattgttccaagacggtccaatgggtttgaagaagtaacccgggctgcaggaattcgatatcaagcttatcgataccgtcgacctcgagtgaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaatcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcataggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcac

SEQ ID NO: 66 - DNA, 8347 bp, synthetic construct
  misc_feature 1..8347: note = pYP106
  source 1..8347: mol_type = other DNA
ctcaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacagttaacatgcatcaccatcaccatcacactagtggatccatgaaggttattaccgcaagagaagctgctgcattggttcaagacggttggaccgttgcttctgctggtttcgttggtgctggtcacgctgaagctgttaccgaagcattggaacaaagattcttgcaatctggattgccaagagacttgaccttggtttactctgctggacaaggtgacagaggtgctagaggtgttaatcacttcggtaacgctggtatgaccgcttctattgttggtggtcactggagatctgctaccagattggctaccttggctatggctgaacaatgtgaaggttacaacttgccacaaggtgttttgacccacctatacagagctattgctggtggtaagccaggtgttatgaccaagattggtttgcacaccttcgttgacccaagaaccgctcaagacgctagataccacggtggtgctgttaatgaaagagctagacaagctatagctgaaggtaaggcttgttgggttgacgctgttgacttcagaggtgacgaatacttgttctacccatctttcccaattcactgtgctttgattagatgtaccgctgctgacgctagaggtaacttgtctacccacagagaagcattccaccacgaattgttggctatggctcaagctgctcacaactctggtggtattgttattgctcaagttgaatctttggttgaccaccacgaaattttgcaagctattcacgttccaggtattttggttgactacgttgttgtttgtgacaacccagctaaccaccaaatgaccttcgctgaatcttacaacccagcttacgttaccccttggcaaggtgaagctgctgttgctgaagctgaagctgctccagttgcagctggtccattggacgctagaaccattgttcaaaggagagctgttatggaattggctagaagggctccaagagttgttaatttgggtgttggtatgccagctgctgttggtatgttggctcaccaagcaggattggacggtttcaccttgaccgttgaagctggtccaattggtggtaccccagctgacggtttgtctttcggagcttctgcttacccagaagctgttgttgaccaaccagctcaattcgacttctacgaaggtggtggtattgacttggctattttgggtttggctgaattggacggtcacggtaacgttaatgtttctaagttcggtgaaggtgaaggtgcttctatagctggtgttggtggtttcattaacattacccaatctgctagagctgttgttttcatgggtaccttgaccgctggtggattggaagttagagctggtgacggtggtttgcaaattgttagagaaggtagagttaagaagattgttcctgaagtttctcacttgtctttcaacggaccatacgttgcttctttgggtattccagttttgtacattaccgaaagagctgttttcgaaatgagagcaggtgctgacggtgaagctagattgaccttggttgaaattgctccaggtgttgacttgcaaagagacgttttggaccaatgttctaccccaattgctgtagctcaagacttgagagaaatggacgctagattgttccaagctggaccattgcacttgtaataacccgggctgcaggaattcgatatcaagcttatcgataccgtcgacctcgaggaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaatcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcataggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgtctattaaatgcttcctatattatatatatagtaatgtcgtttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggag

SEQ ID NO: 67 - DNA, 7645 bp, synthetic construct
  misc_feature 1..7645: note = pYP137
  source 1..7645: mol_type = other DNA
gagctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaagttaacatgcatcaccatcaccatcacactagtggatccatggcagtcctatcctcagctgatagggctagtaacgaaaagaaggtaaagtcatcttacttcgacttgcctccaatggaaatgtcagttgcattcccacaagccacaccagcttctacgtttcccccgtgcacttctgattactatcactttaatgacttgttgacaccagaagagcaggcaattagaaagaaggtaagagagtgtatggaaaaagaagttgctccgattatgactgaatactgggagaaggcagagtttccatttcatataacacctaagctaggggctatgggagttgcaggcggatctatcaaaggttacggttgtccaggcctaagcatcacagccaatgctatcgcaacagccgaaattgcaagggttgatgccagttgttctacgttcattttagtccatagttctttaggaatgctgacaattgctttatgcggtagtgaagcacaaaaagagaaataccttccatccttggcacaacttaatacagtggcctgctgggcgcttactgagccagataatggttctgatgcttcaggattgggaaccacagcgactaaggtggaaggcggttggaagattaacggtcaaaaaaggtggataggaaactcaacattcgcggatttattgattatctttgctagaaacacgactaccaaccaaatcaacggcttcattgtaaagaaagatgctcctggcttaaaagcaaccaaaatccctaataaaattggtttgaggatggtacaaaacggggatatcttgttacagaacgtgtttgtgcccgacgaagatcgtctacccggtgttaattctttccaagacacttccaaggtattagcagtctcacgtgttatggtagcttggcagcctattggtatctctatgggtatctacgatatgtgtcatagatacctgaaagaaaggaagcagtttggagctcctttagctgcttttcaacttaaccagcaaaaattggtacaaatgttaggaaatgttcaagcgatgttccttatgggctggagattgtgtaagttatacgaaactggtcaaatgacacctggtcaggcgtcattaggtaaggcttggatatcctctaaggcaagagaaacagcaagtttgggcagagaacttttaggtggaaacggtattttagccgatttcctagttgcgaaagcattctgtgacttggagcctatatacacatacgaagggacttacgatattaatacattagtgacggggagagaagttacaggcattgctagtttcaaaccagctacaaggtctagattataagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctg

SEQ ID NO: 68 - DNA, 3755 bp, synthetic construct
  misc_feature 1..3755: note = pD902e
  source 1..3755: mol_type = other DNA
cttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagaacaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctgttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattcaaaacgatgtttgcaaaagaaggtgaaaatgaaggttgaaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatcccaccggcgcaattaatatttacttattttggtcaaccccaaataggttgatttcatacttggttcattcaaaaataagtagtcttttgagatctttcaatattataataaatatactataacagccgacttgtttcattttcgcgaatgttcccccagcttatccaccggcgggatc

SEQ ID NO: 69 - DNA, 990 bp, Escherichia coli
  misc_feature 1..990: note = ldhA-sc
  source 1..990: mol_type = genomic DNA
atgaagttggctgtttactctaccaagcaatacgacaagaagtacttgcaacaagttaacgaatctttcggtttcgaattggaattcttcgacttcttgttgaccgaaaagaccgctaagaccgctaacggttgtgaagctgtttgtattttcgttaacgacgacggttctagaccagttttggaagaattgaagaagcacggtgttaagtacattgctttgagatgtgctggtttcaacaacgttgacttggacgctgctaaggaattgggtttgaaggttgttagagttccagcttacgacccagaagctgttgctgaacacgctattggtatgatgatgaccttgaacagaagaattcacagagcttaccaaagaaccagagacgctaacttctctttggaaggtttgaccggtttcaccatgtacggtaagaccgctggtgttattggtaccggtaagattggtgttgctatgttgagaattttgaagggtttcggtatgagattgttggctttcgacccatacccatctgctgctgctttggaattgggtgttgaatacgttgacttgccaaccttgttctctgaatctgacgttatttctttgcactgtccattgaccccagaaaactaccacttgttgaacgaagctgctttcgaacaaatgaagaacggtgttatgattgttaacacctctagaggtgctttgattgactctcaagctgctattgaagctttgaagaaccaaaagattggttctttgggtatggacgtttacgaaaacgaaagagacttgttcttcgaagacaagtctaacgacgttattcaagacgacgttttcagaagattgtctgcttgtcacaacgttttgttcaccggtcaccaagctttcttgaccgctgaagctttgacctctatttctcaaaccaccttgcaaaacttgtctaacttggaaaagggtgaaacctgtccaaacgaattggtttaa

SEQ ID NO: 70 - protein, 329 aa, Escherichia coli
  REGION 1..329: note = ldhA-sc_AA
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 71 - DNA, 7337 bp, synthetic construct
  misc_feature 1..7337: note = pYP024
  source 1..7337: mol_type = other DNA
ctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaaatgaagttggctgtttactctaccaagcaatacgacaagaagtacttgcaacaagttaacgaatctttcggtttcgaattggaattcttcgacttcttgttgaccgaaaagaccgctaagaccgctaacggttgtgaagctgtttgtattttcgttaacgacgacggttctagaccagttttggaagaattgaagaagcacggtgttaagtacattgctttgagatgtgctggtttcaacaacgttgacttggacgctgctaaggaattgggtttgaaggttgttagagttccagcttacgacccagaagctgttgctgaacacgctattggtatgatgatgaccttgaacagaagaattcacagagcttaccaaagaaccagagacgctaacttctctttggaaggtttgaccggtttcaccatgtacggtaagaccgctggtgttattggtaccggtaagattggtgttgctatgttgagaattttgaagggtttcggtatgagattgttggctttcgacccatacccatctgctgctgctttggaattgggtgttgaatacgttgacttgccaaccttgttctctgaatctgacgttatttctttgcactgtccattgaccccagaaaactaccacttgttgaacgaagctgctttcgaacaaatgaagaacggtgttatgattgttaacacctctagaggtgctttgattgactctcaagctgctattgaagctttgaagaaccaaaagattggttctttgggtatggacgtttacgaaaacgaaagagacttgttcttcgaagacaagtctaacgacgttattcaagacgacgttttcagaagattgtctgcttgtcacaacgttttgttcaccggtcaccaagctttcttgaccgctgaagctttgacctctatttctcaaaccaccttgcaaaacttgtctaacttggaaaagggtgaaacctgtccaaacgaattggtttaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcgccagtagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgggaatttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggag